(19) United States
(12) Patent Application Publication
(10) Pub. No.: US 2006/0258604 A1
(43) Pub. Date: Nov. 16, 2006

(54) Compositions and methods for the treatment of inflammatory bowel disease utilizing NF-kappaB decoy polynucleotides

(76) Inventors: Warren Strober, Bethesda, MD (US); Ivan Fuss, Kensington, MD (US); Atsushi Kitani, Rockville, MD (US); Stefan Fichtner-Feigl, Mainburg (DE)

Correspondence Address: NATIONAL INSTITUTE OF HEALTH;C/O NEEDLE & ROSENBERG, P.C., SUITE 1000, 999 PEACHTREE STREET, ATLANTA, GA, 30303, US

(21) Appl. No.: 11/125,919

(22) Filed: May 10, 2005

(51) Int. Cl. A61K 48/00

(52) U.S. Cl. 514/44

(57) ABSTRACT

Provided herein is a method of treating or preventing inflammatory bowel disease (IBD) in a subject comprising administering to the subject a therapeutically effective amount of a composition comprising an NF-κB decoy polynucleotide.

### BACKGROUND OF THE INVENTION

The idiopathic inflammatory bowel diseases (Crohn's disease and ulcerative colitis) are due to inappropriate and/or excessive responses to antigens present in the normal bacterial microflora (1-6). Crohn's disease is characterized by a transmural, granulomatous inflammation occurring anywhere in the alimentary canal, but is usually centered in the terminal ileum and ascending colon; ulcerative colitis, in contrast, is marked by a superficial inflammation causing epithelial cell destruction (ulceration) that is centered in the rectum and colon (1, 2). Despite having a common basis in over-responsiveness to mucosal antigens, the two diseases have considerably different pathophysiologies. Crohn's disease is associated with a Th1 T cell-mediated response induced by IL-12 and possibly IL-23, whereas ulcerative colitis is associated with an atypical Th2-mediated response characterized by NKT cell secretion of IL-13 (6-10).

### BRIEF SUMMARY OF THE INVENTION

Provided herein is a method of treating or preventing inflammatory bowel disease (IBD) in a subject comprising administering to the subject a therapeutically effective amount of a composition comprising an NF-κB decoy polynucleotide.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only, and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the basic properties of NF-κB decoy polynucleotide. (A) Effect of NF-κB decoy polynucleotide on NF-κB DNA-binding activity. HeLa cells activated by TNF-α (20 ng/ml) or Raji cells (constitutively activated) were transfected with NF-κB decoy polynucleotide or scrambled polynucleotide encapsulated in a HVJ-E viral envelope; 30 minutes after stimulation, the binding activity of p65, c-Rel, and p50 was determined in nuclear extracts of HeLa cells, whereas binding activity of Rel B and p52 was directly determined in nuclear extracts of Raji cells using the TransFactor assay. Data shown are mean values±SD obtained from two independent experiments. (B) In vivo transfection of NF-κB decoy polynucleotide into CD4+ T cells and non-CD4+ T cells in the colonic lamina propria. Mice were administered FITC-conjugated NF-κB decoy polynucleotide (or unconjugated NF-κB decoy polynucleotide) 4 h after intra-rectal decoy administration or 4 h, 24 h and 48 h after intra-peritoneal decoy administration; then, 5 days after TNBS-colitis induction, colonic lamina propria cells were isolated, stained with PE-anti-CD4 and analyzed by flow cytometry.

FIG. 2 shows prevention and treatment of TNBS-colitis by administration of NF-κB decoy polynucleotide. (A-D) TNBS-colitis was induced by intra-rectal administration of TNBS in ethanol. Mice were treated with NF-κB decoy polynucleotide (or scrambled polynucleotide) via an intra-rectal route (at 4 h) or via an intra-peritoneal route (at 4 h, 24 h, and 48 h). Data shown are representative of 3 independent experiments. (A) Body weight as a percent of the starting weight. Data shown are mean values±SD and are derived from at least seven mice per group. (B) Animal survival during the first 5 days after TNBS administration. (C) H&E staining of representative colon cross-sections on day 5 after TNBS administration. (D) Histological scores shown are mean values±SD from at least seven mice per group. (E-H) TNBS-colitis was induced by intra-rectal administration of TNBS in ethanol. On day 5 mice with at least 20% body weight loss and not in a recovery phase were pooled and divided into treatment groups. Mice were treated with NF-κB decoy polynucleotide (or scrambled polynucleotide) via an intra-rectal route (day 5) or via an intra-peritoneal route (days 5-7). Data shown are representative of 2 independent experiments. (E) Body weight as a percent of starting weight. Data shown are mean values±SD from at least seven mice per group. (F) Animal survival in percent until day 9 after TNBS administration. (G) H&E staining of representative colon cross-sections on day 9 after TNBS administration. (H) Histological scores shown as mean values±SD from at least seven mice per group.

FIG. 3 shows treatment of established TNBS-colitis with NF-κB decoy polynucleotide—effect on cytokine production, NF-κB activity and T cell apoptosis. (A-C) TNBS-colitis was induced by intra-rectal administration of TNBS in ethanol. On day 5 mice with at least 20% weight loss and not in a recovery phase were pooled and divided into treatment groups. Mice were treated with NF-κB decoy polynucleotide (or scrambled polynucleotide) via an intra-rectal route (day 5) or via an intra-peritoneal route (days 5-7). Data shown are representative of 2 independent experiments. (A) Cytokine production of colonic lamina propria cells on day 9 after TNBS administration. Cells were extracted from the lamina propria and cultured for 48 h in the presence of stimulants (see Examples). Cytokine concentration was determined in the supernatants by ELISA. (B) DNA-binding activity of p65 and c-Rel in nuclear extracts derived from colonic lamina propria cells on day 9 after TNBS administration and measured by the TransFactor Assay. (C) Apoptosis of CD4+ cells in colonic lamina propria one day after intra-rectal treatment of established TNBS-colitis. Mice were treated with NF-κB decoy polynucleotide (or scrambled polynucleotide) on day 5 and colonic lamina propria cells were isolated on day 6 by flow cytometry. Apoptotic cells were determined by Annexin V staining.

FIG. 4 shows treatment of chronic TNBS-colitis by NF-κB decoy polynucleotide. (A-D) Chronic TNBS-colitis was induced by seven weekly intra-rectal administrations of TNBS in ethanol. Mice were treated with NF-κB decoy polynucleotide (or scrambled polynucleotide) via an intra-rectal route (day 37 and day 44) or via an intra-peritoneal route (days 37-39 and days 44-46). (A) Body weight as a percent of starting weight. Data are shown as mean values±SD and are representative of 2 independent experiments. (B) H&E staining of representative colon cross-sections on day 49 after TNBS administration. (C) Masson's trichrome staining of representative colon cross-sections on day 49 after TNBS administration. (D) Collagen content of the colon. Collagen content was determined on day 49 by a Sircol assay. Data shown are mean values±SD and are derived from at least four mice per group.

FIG. 5 shows treatment of chronic TNBS-colitis by NF-κB decoy polynucleotide—Effect on cytokine production and NF-κB binding activity. (A and B) Chronic TNBS-colitis was induced by seven weekly intra-rectal administrations of TNBS in ethanol. Mice were treated with NF-κB decoy polynucleotide (or scrambled polynucleotide) via an intra-rectal route (day 37 and day 44) or via an intra-peritoneal route (day 37-39 and day 44-46). (A) Cytokine production of colonic lamina propria cells on day 49 after the initial TNBS administration. Cells were extracted from the lamina propria and cultured for 48 h in the presence of stimulants (see Examples). Cytokine concentrations were determined in the culture supernatants by ELISA. Data shown are mean values±SD and are representative of two independent experiments. (B) DNA-binding activity of p65 on day 49 after initial TNBS administration in nuclear extracts from colonic lamina propria cells and measured by the TransFactor assay.

FIG. 6 shows prevention of oxazolone-colitis by administration of NF-κB decoy polynucleotide. (A-F) Oxazolone-colitis was induced by intra-rectal administration of oxazolone in ethanol. Mice were treated with NF-κB decoy polynucleotide (or scrambled polynucleotide) via an intra-rectal route (4 h) or via an intra-peritoneal route (4 h, 24 h). Data shown are representative of 2 independent experiments. (A) Body weight in percent of starting weight. Data shown are mean values±SD derived from at least four mice per group and are representative of 2 independent experiments. (B) Animal survival in percent until day 3 after oxazolone administration. (C)H&E staining of representative colon cross-sections on day 3 after oxazolone administration. (D) Histological scores shown are mean values±SD derived from at least four mice per group. (E) Cytokine production of colonic lamina propria cells on day 3 after oxazolone administration. Cells were extracted from the lamina propria and cultured for 48 h in the presence of stimulants. MDC/CCL22 was measured after ex vivo colon culture for 48 h and normalized to 100 mg colon (see Methods). Cytokine concentrations were determined in the supernatant by ELISA. Data are shown are mean values±SD and are representative of two independent experiments. (F) DNA-binding activity of p65 in nuclear extracts of cells derived from the lamina propria on day 3 after oxazolone administration measured in nuclear extracts from colonic lamina propria cells by TransFactor assay.

FIG. 7 shows effects of NF-κB decoy polynucleotide administered via an intra-rectal route on NF-κB binding activity in extra-intestinal mononuclear cells. TNBS-colitis was induced by intra-rectal instillation of TNBS in ethanol. Mice were treated with NF-κB decoy polynucleotide (or scrambled polynucleotide) via intra-rectal route (4 h) or via intra-peritoneal route (4 h, 24 h, 48 h). DNA-binding activity of p65 on day 5 after TNBS administration was measured in nuclear extracts derived from colonic lamina propria mononuclear cells, liver mononuclear cells and splenocytes by TransFactor assay. Data shown are representative of 2 independent experiments involving at least 3 mice in each group.

FIG. 8 shows that TNF-α stimulation leads to apoptosis of NF-κB decoy ODN-transfected CD4⁺ and CD11b⁺ LPMC cells in vitro. Colonic LPMC were separated into CD4⁺ and CD11b⁺ subpopulations by antibody-coated magnetic beads. The cells thus obtained were transfected with NF-κB decoy ODN or scrambled ODN or were left untransfected and then cultured with TNF-α for 24 h. Apoptosis of transfected and untransfected CD4⁺ and CD11b⁺ LPMC was determined by Annexin V and propidium iodide staining at the end of the culture period.

### DETAILED DESCRIPTION OF THE INVENTION

The disclosed methods and compositions may be understood more readily by reference to the following detailed description of particular embodiments and the Example included therein and to the Figures and their previous and following description. It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Provided herein is a method of treating or preventing inflammatory bowel disease (IBD) in a subject comprising administering to the subject a therapeutically effective amount of a composition comprising an NF-κB decoy polynucleotide.

It must be noted that as used herein and in the appended claims, the singular forms “a”, “an”, and “the” include plural reference unless the context clearly dictates otherwise. Thus, for example, reference to “a polynucleotide” includes a plurality of such polynucleotides, reference to “the polynucleotide” is a reference to one or more polynucleotides and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

As used herein, a “subject” includes animals, for example, a vertebrate. More specifically this vertebrate can be a mammal (e.g., a human, horse, pig, rabbit, dog, sheep, goat, non-human primate, cow, cat, guinea pig or rodent (e.g., a rat or mouse)), a fish, a bird or a reptile or an amphibian. The subject may be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treat,” “treating,” or “treatment” is meant a method of reducing the effects of a disease or condition, i.e., IBD. Treatment can also refer to a method of reducing the disease or condition itself rather than just the symptoms. The treatment can be any reduction from native levels and can be, but is not limited to, the complete ablation of the disease (i.e., IBD) or the symptoms of the disease (e.g., inflammation). Treatment can range from a positive change in a symptom or symptoms of IBD (e.g., inflammation, diarrhea, rectal prolapse, weight loss, abdominal pain etc.) to complete amelioration of the inflammatory response of IBD (e.g., reduction in severity or intensity of disease, alteration of clinical parameters indicative of the subject's condition, relief of discomfort or increased or enhanced function), as detected by art-known techniques. For example, a disclosed method is considered to be a treatment if there is about a 10% reduction in one or more symptoms of the disease in a subject with the disease when compared to native levels in the same subject or control subjects. Thus, the reduction can be about a 10, 20, 30, 40, 50, 60, 70, 80, 90, 100%, or any amount of reduction in between as compared to native or control levels.

By “prevent,” “preventing,” or “prevention” is meant a method of precluding, delaying, averting, obviating, forestalling, stopping, or hindering the onset, incidence, severity, or recurrence of a disease, i.e. IBD. For example, the disclosed method is considered to be a prevention if there is about a 10% reduction in onset, incidence, severity, or recurrence of IBD, or symptoms of IBD (e.g., inflammation, diarrhea, rectal prolapse, weight loss, abdominal pain etc.) in a subject with IBD when compared to control subjects. Thus, the reduction in onset, incidence, severity, or recurrence of IBD can be about a 10, 20, 30, 40, 50, 60, 70, 80, 90, 100%, or any amount of reduction in between as compared to control subjects.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

A. Inflammatory Bowel Disease

As described above, the present invention is directed to methods of treating IBD. As utilized throughout, “inflammatory bowel disease” (IBD) refers to a chronic recurrent inflammatory disease of unclear etiology affecting the small intestine and colon that includes both Crohn's disease (CD) and ulcerative colitis (UC). Crohn's disease can involve any portion of the intestinal tract but most commonly involves the distal small intestine and/or the colon. Ulcerative colitis involves only the colon, generally limited to the rectum or distal colon. Studies of murine models of CD and UC strongly suggest that both of these diseases are due to dysregulation of the mucosal immune response to antigens in the mucosal microflora (Sartor, R. B. (1995). Gastroenterol Clin North Am 24, 475-507) (Strober W, et al. (2002) Annu. Rev. Immunol. 20:495-549).

By “inflammatory response” or “immune response” is meant the reaction of living tissues to injury, infection or irritation characterized by redness, warmth, swelling, pain, and loss of function produced, as the result of increased blood flow and an influx of immune cells and secretions. Inflammation is the body's reaction to invading infectious microorganisms and results in an increase in blood flow to the affected area, the release of chemicals that draw white blood cells, an increased flow of plasma, and the arrival of monocytes to clean up the debris. Anything that stimulates the inflammatory response is said to be inflammatory.

One of skill in the art would recognize that ulcerative colitis or indeterminate colitis refers to a condition of the colon characterized by a state of inflammation in which one or more of the following histological characteristics are detectable: a superficial inflammation characterized by the presence of epithelial cell loss and patchy ulceration, pronounced depletion of mucin producing-goblet cells, and reduction of the density of the tubular glands. In addition, in the lamina propia, a mixed inflammatory cell infiltrate consisting of lymphocytes and granulocytes (the latter consisting mostly of neutrophils and, to a lesser extent, eosinophils) associated with an exudation of cells into the bowel lumen is observed. Also, the submucosal level can display marked edema with few inflammatory cells, while in the outer muscle layer one of skill in the art would see little or no evidence of inflammation. See e.g. Boirivant et al. Journal of Experimental Medicine 188: 1929-1939 (1998). Clinical symptoms can include, but are not limited to, diarrhea, rectal prolapse, weight loss, abdominal pain, and dehydration.

Crohn's disease refers to inflammation affecting any part of the alimentary tract but most often affecting the terminal part of the small bowel and/or the adjacent ascending colon. Frequently, the inflammation is characterized by “skip lesions” consisting of areas of inflammation alternating with areas of normal mucosa. The affected area of bowel in Crohn's is marked by erythema, edema and increased friability; at times the bowel is strictured and attached to other abdominal organs or to the bowel wall. Fistulae between the affected bowel and other structures including the skin are not infrequent. Microscopic examination of the tissue in Crohn's disease reveals epithelial erosions, loss of mucin-producing goblet cells and an extensive lymphocytic infiltration involving all layers of the mucosa; this infiltrate sometimes contains giant cells indicative of granuloma formation. When inflammation is present for a long time (chronic), it sometimes can cause scarring (fibrosis). Scar tissue is typically not as flexible as healthy tissue. Therefore, when fibrosis occurs in the intestines, the scarring may narrow the width of the passageway (lumen) of the involved segments of the bowel. These constricted areas are called strictures. The strictures may be mild or severe, depending on how much they block the contents of the bowel from passing through the narrowed area. Clinical signs/symptoms of Crohn's disease can include but are not limited to: cachexia, weight loss, poor growth, abdominal pain, draining fistulae, rectal prolapse and dehydration.

Thus, the herein provided methods, comprising administering to a subject a therapeutically effective amount of a composition comprising an NF-κB decoy polynucleotide, can be used to treat or prevent ulcerative colitis. Further, the herein provided methods, comprising administering to a subject a therapeutically effective amount of a composition comprising an NF-κB decoy polynucleotide, can be used to treat or prevent Crohn's disease, including chronic Crohn's disease.

Further provided by the present invention is a method of ameliorating a Th2 inflammatory response associated with inflammatory bowel disease in a subject comprising administering to the subject a therapeutically effective amount of a composition comprising an NF-κB decoy. Also provided is a method of ameliorating a Th1 inflammatory response associated with inflammatory bowel disease in a subject comprising administering to the subject a therapeutically effective amount of a composition comprising an NF-κB decoy.

In order to treat or prevent inflammatory bowel disease, the methods of the present invention comprise the delivery of an NF-κB decoy polynucleotide to the nucleus of gastrointestinal cells in a subject. In one aspect, a NF-κB decoy polynucleotide can be delivered to any cell within the gastrointestinal tract. As the NF-κB decoy polynucleotides of the methods provided herein is used herein to reduce inflammation of IBD, the preferred target cells are those expressing NF-κB and contributing to the inflammatory response. Thus, the methods provided herein comprise the delivery of an NF-κB decoy polynucleotide to the nucleus of, for example, epithelial cells, antigen presenting cells, B-cells, T-cells, macrophages, monocytes, eosinophils, fibroblasts, myofibroblasts, and neutrophils.

B. NF-κB Decoys

As described above, the present invention is directed to the use of NF-κB decoy polynucleotides to treat or prevent IBD. The major family of NF-κB transcription factors consists of five members, c-Rel, p65, Rel B, p50, and p52, all of which contain domains that bind to a similar binding site in the promoters of genes encoding key inflammatory proteins (such as, but not limited to, IL-12 and IL-23) (28). Each of these NF-κB transcription factors binds to a chromosomal NF-κB binding site and activates transcription of a gene located downstream of the NF-κB binding site. Each transcription factor can bind to one or more NF-κB binding sites, thus activating transcription of one or more genes. Therefore, the NF-κB decoys of the present invention mimic chromosomal NF-κB binding sites, thus allowing NF-κB transcription factors to bind to the NF-κB decoy. Therefore, the NF-κB decoys of the present invention inhibit the binding of NF-κB transcription factors to chromosomal NF-κB binding sites, thus inhibiting transcription of genes located downstream from the NF-κB binding sites. In other words, once an NF-κB decoy is introduced into cells, the decoy specifically binds to NF-κB transcription factors, thereby, inhibiting transcription of target genes by these transcription factors.

By “inhibit,” “inhibiting,” and “inhibition” is meant a decrease in an activity, response, condition, disease, or other biological parameter. This can include but is not limited to the complete ablation of the activity, response, condition, or disease. This may also include, for example, about a 10% reduction in the activity, response, condition, or disease as compared to the native or control level. Thus, the reduction can be about a 10, 20, 30, 40, 50, 60, 70, 80, 90, 100%, or any amount of reduction in between as compared to native or control levels.

The NF-κB decoys provided herein and utilized in the methods described herein can comprise a polynucleotide, or an analog thereof. The polynucleotide can be DNA or RNA, complements thereof, and can contain modified nucleotides and/or pseudonucleotides. Furthermore, the polynucleotide can be single-stranded or double-stranded. Also, the polynucleotide can be linear or cyclic. Thus, the NF-κB decoy of the provided methods can be an oligonucleotide, including oligodeoxynucleotide or oligoribonucleotide. Variants that can exist in the disclosed polynucleotide include mutations such as substitutions, additions and/or deletions of any part of the above sequence, wherein the variant is still able to specifically antagonize or inhibit the binding of NF-κB to promoter sites on chromosomes. Thus, in one aspect, the NF-κB decoy of the present method is a double-stranded DNA polynucleotide, comprising one or a plurality of the above nucleotide sequence and variants thereof. The polynucleotide that can be used in the present method includes polynucleotides modified so as to be less susceptible to biodegradation, such as those polynucleotides containing a thiophosphoric diester bond available upon substitution of sulfur for the oxygen of the phosphoric diester moiety (S-oligo) and those polynucleotides available upon substitution of a methyl phosphate group carrying no electric charge for the phosphoric diester moiety.

The NF-κB decoy of the present method can be any polynucleotide comprising a NF-κB binding sequence that an NF-κB transcription factor can bind to. As used herein, the term “binds” or “binding”, when referring to a DNA:polypeptide interaction, refers to the ability of the polypeptide to bind to a specified cis element, for example an NF-κB binding sequence, but not to wholly unrelated nucleic acid sequences. Such binding may be by any chemical, physical or biological interaction between the cis element and the polypeptide, including, but not limited, to any covalent, steric, agostic, electronic and ionic interaction between the cis element and the polypeptide. A variety of well-known techniques can be used to identify DNA:polypeptide binding, e.g., mobility shift DNA-binding assays, methylation and uracil interference assays, DNase and hydroxy radical footprinting analysis, fluorescence polarization, and UV crosslinking or chemical cross-linkers.

The NF-κB decoy polynucleotides of the present invention can be derived from the promoter of a gene comprising an NF-κB binding site. Genes that comprise a NF-κB binding site include, but are not limited to, 11bHSD2, 25-hydroxyvtamin D3 1alpha hydroxylase, A1 adenosine receptor, a1-antitrypsin, A20, ABC Transporters, Adenovirus (E3 region), ADH, alpha 1ACT, alpha-1 acid glycoprotein, alpha2B-adrenergic receptor, alpha-fetoprotein, AMH, Amiloride-sensitive sodium channel, Androgen receptor, Angiopoietin, Angiotensin II, Angiotensinogen, Apolipoprotein C III, Apolipoprotein E, ARF-related protein-1, Aromatase (promoter II), Avian Leukosis Virus, b2 Microglobulin, B94, BACE, Bcl-xL, beta-amyloid, beta-defensin-2, Bfl1/A1, Biglycan, BMP-2, Bovine Leukemia Virus, Bradykinin B1-Receptor, BRCA2, BRL-1, C4b binding protein, Caspase-11, Cathepsin B, Cathepsin L, Caveolin-1, CCL15/Leukotactin, CCL22, CCR5, CCR7, CD137, CD23, CD48, CD69, CD83, Ceramide glycosyltransferase, c-FLIP, c-fos (fish gene), CINC-1, cis-retinoid/androgen dehydrogenase type 1 (CRAD1), cis-retinoid/androgen dehydrogenase type 2 (CRAD2), CMV, c-myb, c-myc, Complement B, Complement component 3, Complement factor B, Complement factor C4, Complement Receptor 2, Connexin32, C-reactive protein, c-rel, Cu/Zn SOD, Cyclin D1, Cyclin D2, Cyclin D3, CYP2C11, CYP2E1, DC-SIGN, Dihydrodiol dehydrogenase, DNASIL2, DYPD, E2F3a, EBV (Wp promoter), Egr-1, ELAM-1 (CD62E, E-selectin), Elf3, ELYS, Endoglin, ENO2, Ephrin-A1, Epidermal Growth Factor Receptor, EPO, epsilon-Globin, ETR101, Factor VIII, Fas-associated phosphatase-1, Fc epsilon receptor II (CD23), Ferritin H chain, GAD67, Gadd45beta, Gal1 Receptor, Galectin 3, Galpha i2, GBP-1, GD3-synthase, Gelatinase B, GIF, Glucocorticoid receptor, Glucose 1-6-phosphate dehydrogenase, Glutamate-cysteine ligase, Glutamate-cysteine ligase modifier, Gro a-g, Gro-1, GS3686, GSTP1-1, H+-K+ATPase alpha2, HBV (pregenomic promoter), Heparanase, HIV-1, HMG14, HO-1, HPV type 16, HSV, Hyaluronan synthase, ICOS, IEX-1L, IGFBP-1, IGFBP-2, IkB-a, IL-10, IL-2 receptor a-chain, IL-9, Immunoglobulin Cgamma1, Immunoglobulin e heavy chain, Immunoglobulin gamma4, Immunoglobulin k light chain, Inducible NO-Synthase, Invariant Chain I_{I}, Iodothyronine deiodinase (type 2), JC Virus, junB, K15 Keratin, K3 Keratin, K6 Keratin, KC, Laminin B2 Chain, Lipocalin-type prostaglandin D synthase (L-PGDS), Lipopolysaccharide binding protein, LIX (mouse), ENA-78 (CXCL5), GCP-2 (CXCL6) (human), Lox-1, Lymphotoxin a, Lymphotoxin b, Lysozyme, Mail, MAP4K1, M-CSF (CSF-1), Mdr1, MHC class I (H-2 Kb), MHC Class I HLA-B7, MIP-2, MKP-1, MMP-3, matrix metalloproteinaase-3, MMP-9, matrix metalloproteinaase-9, Mn SOD, MNE1, mob-1, Mts1, Mucin (MUC-2), Mu-opioid receptor, Mx1 (bovine), N-acetylglucosaminyltransferase I (rat gene), NAD(P)H quinone oxidoreductase (DT-diaphorase), Neuropeptide Y-Y1 receptor, Neutrophil activating peptide-78, Neutrophil gelatinase-associated lipocalin, nfkb1, nfkb2, NK-1R, NK4, NLF1, NMDA receptor subunit 2A (rat), NMDA receptor subunit NR-1 (GRIN1 gene), Nr13, NURR1, p11, p21-CIP1, p22/PRG1, p53, p62, PAF receptor 1, PAI-1, Pax8, PCBD, PDE7A1, PDGF B chain, Pentraxin PTX3, Peptide Transporter TAP1, Perforin, PGES, prostaglandin E synthase, PGK1, PIM-1, PKCdelta, PLCdelta 1, Plk3, Polymeric Ig receptor, POMC, PP5, Pregnancy-specific glycoprotein mCGM3, Prodynorphin, Proenkephalin, Prostate-specific antigen, Proteasome Subunit LMP2, P-selectin, PTGIS, prostaglandin synthase, RACK1, RAGE-receptor for advanced glycation end products, relb, REV3, S100A6 (calcyclin), Serpin 2A, Serum amyloid A proteins (SAA1, SAA2, SAA3), SIAT1, SIV, Snail, SNARK, Soluble Guanylyl cyclase alpha (1), Sox9, Spergen-1, Stat5a, Stem Cell Factor, SV-40, SWS1, Syndecan-4, Tapasin, TCA3, T-cell activation gene 3, T-cell receptor b chain, T-cell receptor/CD3gamma, Tenascin-C, TERT (mouse), TFF3 (Treefoil factor), Thrombospondin-1 (TSP-1), Thrombospondin-2 (THBS2), TIEG, Tissue factor pathway inhibitor-2 (TFPI-2), Tissue factor-1, TLR-2, TLR9, TNFbTRAF-1, TRAF-2, TRAIL (aka Apo2 ligand), Transferrin (mosquito), Transglutaminase, TRIF, Type II-secreted phospholipase A2, UBE2M, UCP-2, Urokinase-type plasminogen activator, Uroplakin Ib, VEGF C, VEGI, Vimentin, WT1, and Xanthine Dehydrogenase.

Examples of genes involved in inflammatory disease that comprise a NF-κB binding site include 12-Lipoxygenase, 5-Lipoxygenase, (I) collagen, B7.1 (CD80), Bax, Bcl-2, b-Interferon, CCL28, CCL5, CD154, CD40, CD95 (Fas), Claudin-2, Collagenase 1, COX-2, CXCL 11, Eotaxin, Fas-Ligand, Fibronectin, Fractalkine, G-CSF, GM-CSF, HGF/SF, IAPs, ICAM-1, IFN-g, IL-1 receptor antagonist, IL-11, IL-12 (p40), IL-12 (p35), IL-13, IL-15, IL17, IL23 (p19), IL-1a, IL-1b, IL-2, IL-6, IL-8, iNOS, IP-10, IRF-1, IRF-2, IRF-4, IRF-7, MadCAM-1, MCP-1/JE, MIP-1a,b (LAG-1), MIP-3alpha, MIG, Nod2, Phospholipase A2, RANTES, RICK, TNFa, TNF-Receptor (p75/80,CD120B), and VCAM-1. Thus, the NF-κB decoy can be a polynucleotide comprising the NF-κB binding site of a gene encoding 12-Lipoxygenase, 5-Lipoxygenase, (I) collagen, B7.1 (CD80), Bax, Bcl-2, b-Interferon, CCL28, CCL5, CD154, CD40, CD95 (Fas), Claudin-2, Collagenase 1, COX-2, CXCL 11, Eotaxin, Fas-Ligand, Fibronectin, Fractalkine, G-CSF, GM-CSF, HGF/SF, IAPs, ICAM-1, IFN-g, IL-1 receptor antagonist, IL-11, IL-12 (p40), IL-12 (p35), IL-13, IL-15, IL17, IL23 (p19), IL-1a, IL-1b, IL-2, IL-6, IL-8, iNOS, IP-10, IRF-1, IRF-2, IRF-4, IRF-7, MadCAM-1, MCP-1/JE, MIP-1a,b (LAG-1), MIP-3alpha, MIG, Nod2, Phospholipase A2, RANTES, RICK, TNFa, TNF-Receptor (p75/80,CD120B), or VCAM-1.

One of skill in the art can determine if overexpression of one or more of these genes is associated with inflammatory bowel disease, for example, by any of the methods known in the art for assessing differential gene expression, such as microarray techniques. Once these genes are identified, their NF-κB binding sites can be aligned and a consensus NF-κB binding sequence consisting essentially of nucleotides shared by the NF-κB binding sites of the differentially expressed genes can be obtained. For a description of how to identify differentially expressed genes and analyze transcription factor binding sites, please see US Patent Application Publication No. 20040191779, published Sep. 30, 2004 which is hereby incorporated by this reference in its entirety for its teachings regarding differential gene expression and analysis of transcription factor binding sites and the determination of consensus sequences of differentially expressed genes.

In one aspect of the herein provided method, the NF-κB decoy polynucleotide specifically binds one or more NF-κB subunits selected from the group consisting of NF-κB1 (p50, p105), NF-κB2 (p52, p100), RelA (p65), RelB, or c-Rel bind. Thus, the NF-κB decoy polynucleotide can specifically bind 1, 2, 3, 4, or 5 of the NF-κB subunits. Thus, in one aspect, the NF-κB decoy polynucleotide can specifically bind p52, p65, RelB, and c-Rel, but not p50. In another aspect, the NF-κB decoy polynucleotide can specifically bind p50, p52, RelB, and c-Rel, but not p65. In another aspect, the NF-κB decoy polynucleotide can specifically bind p52, RelB, and c-Rel, but not p50 or p65. These examples are not meant to be limiting and are merely exemplary of the combinations of NF-κB transcription factors than can bind to an NF-κB decoy.

In another aspect, NF-κB activity can be blocked with a single NF-κB decoy polynucleotide comprising a consensus sequence derived from one or more NF-κB binding sites. Thus, disclosed herein is a NF-κB decoy polynucleotide comprising an NF-κB consensus binding site. A consensus sequence is derived or created by picking the most frequent base at one more positions in a set of aligned DNA or RNA nucleic acid sequences comprising an NF-κB binding site.

As shown in Table 1, the NF-κB consensus binding site can consist essentially of the nucleic acid sequence GGGDNWTTCC, wherein N can be G, A, C, or T, D can be G, A, or T; and W can be A or T (SEQ ID NO:25). The NF-κB consensus binding site can also consist essentially of the nucleic acid sequence GGGATTTCC (SEQ ID NO:5). As shown in Table 2, the NF-κB consensus binding site can also consist essentially of the nucleic acid sequence GGGRNWTTCC (SEQ ID NO:9), wherein R can be G or A; N can be any nucleotide, and W can be A or T (see Chen F E and Ghosh G. Regulation of DNA binding by Rel/NF-kappaB transcription factors: structural views. Oncogene. 1999 Nov. 22; 18(49):6845-52, hereby incorporated herein by reference for its teaching of NF-κB consensus binding sequences).

TABLE 1
NF-κB consensus binding site.

| | | R = G or A |
|---|---|---|
| | | N = any |
| | | W = A or T |
| NF-κB subunits | Binding site | D = G, A, or T |
| Consensus 1 | GGGDNWTTCC | SEQ ID NO:25 |
| Consensus 2 | GGG-ATTTCC | SEQ ID NO:5 |
| p65 | GGGGTATTTCCC | SEQ ID NO:6 |
| c-REL | GGGGTATTTCC | SEQ ID NO:7 |
| p50 | GGGG-AT--CCC | SEQ ID NO:8 |
| Rel B | GGGGTATTTCC | SEQ ID NO:7 |
| p52 | GGGGTATTTCC | SEQ ID NO:7 |

TABLE 2
NF-κB p50/p65 heterodimer binding sites.

| | | R = G or A |
|---|---|---|
| | | N = any |
| | | W = A or T |
| NF-κB subunits | Binding site | D = G, A, or T |
| Consensus 1 | GGGDNWTTCC | SEQ ID NO:25 |
| Consensus 2 | GGG-ATTTCC | SEQ ID NO:5 |
| Consensus 3 | GGGRNWTTCC | SEQ ID NO:9 |
| Igκ | GGGACTTTCC | SEQ ID NO:1O |
| H2 | GGGGATTCCC | SEQ ID NO:11 |
| IFN-β | GGGAAATTCC | SEQ ID NO:12 |
| LCAM | GGGGATTTCC | SEQ ID NO:13 |
| IL-6 | GGGATTTTCC | SEQ ID NO:14 |
| E-selectin | GGGGATTTCC | SEQ ID NO:15 |
| TCR-β | GGGAGATTCC | SEQ ID NO:16 |
| Lymphotoxin | GGGGGCTTCC | SEQ ID NO:17 |
| TNFα | GGGGCTTTCC | SEQ ID NO:18 |
| VCAM | GGGGTTTCCC | SEQ ID NO:19 |
| Angio- | GGGATTTCCC | SEQ ID NO:20 |
| tensinogen | | |
| IL-2R | GGGAATTCCC | SEQ ID NO:21 |
| IL-2 | GGGATTTCAC | SEQ ID NO:22 |
| GM-CSF | GGGAACTACC | SEQ ID NO:23 |
| Urokinase | GGGAAAGTAC | SEQ ID NO:24 |

Thus, the NF-κB decoy of the provided method can be any polynucleotide comprising the nucleic acid sequence SEQ ID NO:25, SEQ ID NO:5 or SEQ ID NO: 9. Further, the NF-κB decoy of the provided method can be any polynucleotide comprising a nucleic acid sequence with at least 80%, 85,%, 90%, 95% sequence identity to SEQ ID NO:25, SEQ ID NO:5 or SEQ ID NO: 9. Likewise, the NF-κB decoy of the provided method can comprise a polynucleotide that hybridizes under stringent or highly stringent conditions to a polynucleotide comprising the sequence set forth as SEQ ID NO:25, SEQ ID NO:5 or SEQ ID NO: 9.

The NF-κB decoy polynucleotide can comprise 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more nucleotides. Thus, the NF-κB decoy of the provided method can be any isolated nucleic acid comprising the sequence SEQ ID NO:25, SEQ ID NO:5 or SEQ ID NO: 9, flanked on either or both the 5′ and 3′ ends with one or more, including 1, 2, 3, 4, 5, 6, 7, 8, 9, 10, 11, 12, 13, 14, 15, 16, 17, 18, 19, or 20, nucleotides. The NF-κB decoy polynucleotide can comprise tandem repeats of NF-κB consensus binding sites. The NF-κB consensus binding sites can be separated by nucleic acid spacer sequences. The spacer sequences can comprise one or more nucleotides, including 1, 2, 3, 4, 5, 6, 7, 8, 9, 10, 11, 12, 13, 14, 15, 16, 17, 18, 19, or 20 nucleotides.

Thus, the NF-κB decoy of the provided method can be any isolated nucleic acid comprising the nucleic acid sequence X₁-C₁-X₂; X₁-C₁-X₂-C₂-X₃; X₁-C₁-X₂-C₂-X₃-C₃-X₄; or X₁-C₁-X₂-C₂-X₃-C₃-X₄C₄-X₅. wherein C, including C₁, C₂, etc., can be Consensus 1 (SEQ ID NO:25), Consensus 2 (SEQ ID NO:25), or Consensus 3 (SEQ ID NO:9); and X, including X₁, X₂, etc., can be none, one or more, including 0, 1, 2, 3, 4, 5, 6, 7, 8, 9, 10, 11, 12, 13, 14, 15, 16, 17, 18, 19, or 20, nucleotides. The nucleic acid sequence of flanking or spacer sequences (i.e., X₁, X₂, etc.) is generally random, and preferably does not comprise tandem repeats or other sequences known in the art to result in secondary polynucleotide structures. Examples of NF-κB decoy polynucleotides comprising flanking and spacer sequences are given in Table 3, using NF-κB Consensus 2 (SEQ ID NO:5) for exemplification. Thus, in one aspect, the NF-κB decoy polynucleotide comprises the nucleic acid sequence CCTTGAAGGGATTTCCCTCC (SEQ ID NO:1).

TABLE 3
NF-κB Decoy Polynucleotides

NF-κB Decoy Polynucleotide

| | |
|---|---|
| ccttgaaGGGATTTCCctcc | SEQ ID NO:1 |
| ccttgaaGGGATTTCC | SEQ ID NO:26 |
| GGGATTTCCgtctaga | SEQ ID NO:27 |
| GGGATTTCCaggatcagaaGGGATTTCC | SEQ ID NO:28 |
| GGGATTTCCagtactggcaGGGATTTCCctcc | SEQ ID NO:29 |
| agtcGGGATTTCCgtccatgatcGGGATTTCC | SEQ ID NO:30 |
| ctgaGGGATTTCCgactagtcatGGGATTTCCatac | SEQ ID NO:31 |
| GGGATTTCCagtacatgcgGGGATTTCCgatctgatagGGGATTTCC | SEQ ID NO:32 |

C. Nucleic Acids

The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that in some cases it is advantageous that a polynucleotide be made up of nucleotide analogs that reduce the degradation of the polynucleotide in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, Ni, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or 0) at the C6 position of purine nucleotides.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

D. Sequence Similarities

It is understood that as discussed herein the use of the terms homology and sequence identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed nucleic acids herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two nucleic acids such as polynucleotides or genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

E. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987: 154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

F. Delivery of the Polynucleotides to Cells

There are a number of compositions and methods which can be used to deliver nucleic acids to cells, such as gastrointestinal cells, in a subject with IBD. The nucleic acids can be delivered by, for example, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. Further, these methods can be used to target certain diseases and cell populations by using the targeting characteristics of the carrier.

In one aspect of the herein provided method, NF-κB decoy polynucleotide is delivered to a cell as naked DNA. Thus, the disclosed polynucleotide can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

In another aspect, NF-κB decoy polynucleotide is delivered to a cell in a lipid carrier. Thus, the compositions can comprise, in addition to the disclosed NF-κB decoy polynucleotide, lipids such as liposomes, for example, cationic liposomes (e.g., DOTMA, DOPE, DC-cholesterol) or anionic liposomes. Liposomes can include commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In another aspect, a NF-κB decoy polynucleotide is delivered to a cell in a chimeric vector. Examples of chimeric systems include pseudotyped retrovirus vector with vesicular stomatitis virus (VSV) G-protein envelope and a lentivirus vector containing HIV proteins decorated with pseudotype retrovirus envelope-containing VSV-G protein (33). An example of non-viral chimeric vectors is a cationic lipid-protamine-DNA complex (33). Further, chimeric gene transfer systems have been developed comprising combinations of viral and non-viral features. For example, DNA-loaded liposomes have been combined with a fusigenic envelope derived from hemagglutinating virus of Japan (HVJ; Sendai virus) (33). Polynucleotides can be incorporated into liposomes with this system and delivered efficiently both in vitro and in vivo by virus-cell fusion, which protects the contents from degradation by endosomes and lysosomes. Thus, NF-κB decoy nucleic acid can be packaged in a chimeric liposome comprising viral envelope-derived fusion (fusigenic) proteins. As an example, NF-κB decoy nucleic acid can be packaged in a HVJ-liposome complex (Dainippon).

NF-κB decoy polynucleotide can also be delivered to a cell in a viral envelope, i.e., encapsulated, or in a viral capsid, i.e., encapsidated. A method for converting the lipid envelope of an inactivated virus to a gene transfer vector is described in Kaneda, Y., et al. (Mol Ther. 2002. August; 6(2):219-26), which is hereby incorporated herein by reference in its entirety for the teaching of HVJ-E vectors and uses thereof. Therein, hemagglutinating virus of Japan (HVJ; Sendai virus) envelope vector was constructed by incorporating plasmid DNA into inactivated HVJ particles. This HVJ envelope vector introduces plasmid DNA into cells efficiently and rapidly. Further, the injection of HVJ envelope vector in vivo results in gene expression in organs including intestine, liver, brain, skin, uterus, tumor masses, lung, spleen, and eye. Thus, in another aspect of the herein provided method, NF-κB decoy polynucleotide is delivered to a cell in a fusigenic viral envelope derived from HVJ-E. Also considered is the use of other fusigenic viral envelopes, such as those derived from other members of the Sendai virus family of viruses.

In another aspect, NF-κB decoy polynucleotide is delivered to a cell in a transfer vector. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

In one aspect, the NF-κB decoy polynucleotide is delivered to a cell in a single-stranded DNA (ssDNA) expression vector. The essential elements for a ssDNA expression vector include 1) a gene encoding a functional reverse transcriptase (RT), 2) a primer binding site for RT initiation, 3) the desired coding sequence, and 4) a stem-loop structure proximal to the coding sequence for the termination of the RT reaction. An example of a ssDNA expression vector is given by Chen Y and McMicken H W (Gene Ther. 2003 September; 10(20):1776-80, hereby incorporated herein by reference for the teaching of a ssDNA expression vector). In addition, bacterial retrons can be used to express ssDNA. Bacterial retrons, isolated from Gram-negative bacteria such as Myxococcus xanthus, Stigmatella aurantiaca, and Escherichia coli, which are the genetic elements responsible for the synthesis of multi-copy single-stranded DNA (msDNA) (Miyata S, et al. Proc Natl Acad Sci USA. 1992 Jul. 1; 89(13):5735-9; Mirochnitchenko O, et al. J Biol. Chem. 1994 Jan. 28; 269(4):2380-3; Mao J R, et al. J Biol. Chem. 1995 Aug. 25; 270(34):19684-7; Lampson B, et al. Prog Nucleic Acid Res Mol. Biol. 2001; 67:65-91, which are hereby incorporated herein by reference for the teaching of msDNA expression vectors.)

As used herein, plasmid or viral vectors can be used to transport the disclosed nucleic acids, such as NF-κB decoys into a cell without degradation. In one aspect, the NF-κB decoy sequence is functionally linked to an expression control sequence (i.e., promoter). A promoter is generally a sequence or sequences of DNA that function when in a relatively fixed location in regard to the transcription start site. A promoter contains core elements required for basic interaction of RNA polymerase and transcription factors, and may contain upstream elements and response elements. As an example, in a ssDNA expression vector, a promoter drives the expression of an mRNA comprising the RT and NF-κB decoy sequences separated by a stem-loop. Viral vectors include, for example, Adenovirus, Adeno-associated virus, Herpes virus, Vaccinia virus, Polio virus, AIDS virus, neuronal trophic virus, Sindbis and other RNA viruses, including viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)), the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The disclosed vectors thus provide DNA molecules which are capable of integration into a mammalian chromosome without substantial toxicity.

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

G. Administration

The substances provided herein can be administered in a number of ways depending on whether local or systemic treatment is desired, and on the area to be treated. The disclosed substances can be administered, for example, orally, intravenously, by inhalation, intranasally, intrarectally, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical (i.e., intrarectal) administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration include powders or granules, suspensions or solutions in water or non-aqueous media, capsules, sachets, or tablets. Thickeners, flavorings, diluents, emulsifiers, dispersing aids or binders can be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The substances provided herein can be delivered at effective amounts or concentrations. An effective concentration or amount of a substance is one that results in treatment or prevention of the inflammatory response of IBD. Effective dosages and schedules for administering the provided substance can be determined empirically, and making such determinations is within the skill in the art. Those skilled in the art will understand that the dosage of the provided substances that must be administered will vary depending on, for example, the subject that will receive the substance, the route of administration, the particular type of substance used and other drugs being administered. One of skill in the art can utilize in vitro assays to optimize the in vivo dosage of a particular substance, including concentration and time course of administration.

The dosage ranges for the administration of the substances are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

A typical daily dosage of the provided substance might range from about 1 μg/kg to up to 100 mg/kg of body weight or more per day, depending on the factors mentioned above. In one aspect, treatment can consist of a single/daily dosage of 1 mg to 20 mg/kg of body weight of a substance provided herein. In another aspect, the substance is infused during a period from 10 minutes to 48 hours. As another example, NF-κB decoy polynucleotides provided herein can be administered at about 1, 2, 3, or 4 mg/kg. Thus, in one aspect, the NF-κB decoy polynucleotides provided herein are administered intrarectally at about 1, 2, 3, or 4 mg/kg per day. In another aspect, the NF-κB decoy polynucleotides provided herein are administered intraperitoneally at about 1, 2, 3, or 4 mg/kg three times per day.

The blood pressure, pulse and temperature of the subjects can be monitored prior to and at 30 minute intervals during the two hour infusion period. Subjects can be given a laboratory evaluation consisting of a complete blood count (CBC) with differential, platelet count, SMA-18 chemistry profile, erythrocyte sedimentation rate (ESR) and a C-reactive protein assay at 1) the time of infusion; 2) 24 hours after infusion; 3) 72 hours after infusion; 4) two weeks after the last infusion; 5) four weeks after the last infusion; (6) six weeks after the last infusion; and 7) eight weeks after the last infusion.

Subjects can also undergo routine colonoscopy with video surveillance at the time of the infusion of a substance provided herein and again at two, four, six and eight weeks after the last infusion. Additionally, serum samples from the subjects can be assayed by ELISA for an inflammatory cytokine(s) (e.g., IL-12, IL-13, IL-23, etc.) levels to monitor drug efficacy. Also, tissue biopsy samples obtained during colonoscopy can be cultured for purified, isolated lamina propia cells and assayed as well. Purified PBM can also be isolated, cultured and assayed.

For example, to evaluate the efficacy of treatment of humans with IBD, such as for example ulcerative colitis or Crohn's disease, with NF-κB decoy polynucleotides, the following studies can be performed. Patients with active inflammation of the colon and/or the terminal ileum who have failed standard medical therapy, which can include prednisone and/or other immunomodulators known in the art (parenterally or orally) for control of IBD can be selected. Drug efficacy can be monitored via colonoscopy. Patients can be randomized to two different protocols. In one protocol, subjects can remain on initial medication and in the second protocol, subjects can have their medication tapered after receiving the NF-κB decoy polynucleotides.

Following administration of a substance for treating, inhibiting, or preventing IBD, the efficacy of the therapeutic substance can be assessed in various ways well known to the skilled practitioner. For instance, one of ordinary skill in the art will understand that a substance provided herein is efficacious in treating or inhibiting inflammation of an established IBD in a subject by observing that the substance reduces inflammation or prevents a further increase in inflammation. Inflammation can be measured by methods that are known in the art, for example, using tissue biopsies to assess tissue damage or antibody assays (e.g., ELISA) to detect the presence of inflammatory cytokines in a sample (e.g., bodily fluids, but not limited to, blood) from a subject or patient, or by measuring the cytokine levels in the patient.

The substances provided herein can be administered prophylactically to patients or subjects who are at risk for having IBD or who have been newly diagnosed with IBD. In subjects who have been newly diagnosed with IBD but who have not yet displayed an established colitis or the inflammatory response of an established colitis (as measured by biopsy or other assays for detecting the inflammation due to colitis) in blood or other body fluid, efficacious treatment with an substance provided herein partially or completely inhibits the appearance of IBD symptoms and/or onset of UC or CD.

H. Co-Administration

Also disclosed are methods for the treatment or prevention of the inflammatory response of IBD comprising co-administratering any of the herein provided substances with another therapeutic agent. Other therapeutic agents can include, but are not limited to, antibodies, soluble receptors, modified ligands, cytokines, or immunomodulatory agents.

Examples of these cytokines, antibodies and immunomodulatory agents that can be employed in the methods provided herein include, but are not limited to, Azathioprine, 6-mercaptopurine, methotrexate, IVIG, IFNα, IFNβ, TNFα Inhibitors (e.g., Enbrel® (entanercept), Remicade® (infliximab) and Humira® (adalimumab)), antisera against lymphocyte membrane antigens (i.e. anti-thymocyte serum (ATS), anti-thymocyte globulin (ATG), anti-lymphocyte serum (ALS), anti-lymphocyte globulin (ALG), anti-CD3, anti-CD4, anti-CD8, anti-IL-4, anti-α^{E}β₇, anti-α⁴β₇, anti-IL-12, or anti-IL-13), anti-TNFα, anti-IFNγ, antisense STAT4 oligonucleotides, anti-ICAM1, antisense ICAM-1 oligonucleotides, anti-CD40L, anti-CD25 (anti-Tac), and IL-10. Examples of soluble receptors that can be employed in the methods provided herein include, but are not limited to, IL-13Rα-Fc and IL-13Rα2-Fc. Examples of modified ligands that can be employed in the methods provided herein include, but are not limited to hIL13 linked to pseudomonas exotoxins (hIL13PE) (e.g. hIL13PE35, hIL13PE38, hIL13PE38 KDEL, hIL13PE40, hIL13PE4E, and hIL13PE38QQR) and mutant hIL13 ligands that compete for IL-13 receptor binding (e.g., hIL-13E13K).

Also disclosed are methods for the treatment or prevention of the inflammatory response of IBD comprising combining the use of any of the herein provided substances with extracorporeal therapies such as leukocytapheresis and extracorporeal photopheresis (ECP). Extracorporeal therapies are effective for IBDs through immunomodulation, such as decrease in circulating activated T-lymphocytes and activated granulocytes that play a central role in the pathogenesis of IBD. ECP is a leukapheresis-based immunomodulatory therapy that has been approved by the US Food and Drug Administration for the treatment of cutaneous T-cell lymphoma (CTCL) since 1988. ECP proceeds as follows: A 16-gauge peripheral intravenous line or a temporary central venous access is placed in the patient. Patients undergo discontinuous leukapheresis of 240 mL of leukocyte-enriched blood. This sample (constituting 25-50% of peripheral blood mononuclear cells) is mixed with 300 mL plasma, 200 mL sterile saline, 5000 U heparin, and 200 mcg 8-methoxypsoralen (UVADEX®), which makes the T-lymphocytes more sensitive to ultraviolet (UV) light, more specifically the long wavelength form called UV-A. The preparation is passed as a 1-mm film through a sterile cassette surrounded by UV-A bulbs for 180 minutes, resulting in an average UV-A exposure of 2 J/cm² per lymphocyte. The mixture is returned to the patient; intravenous access is discontinued. The entire procedure is completed within approximately 4 hours.

Combinations of the cytokines, antibodies, soluble receptors, immunomodulatory agents, and extracorporeal therapies disclosed herein can also be administered to a subject with an NF-κB polynucleotide of the present invention.

Other antibodies, soluble receptors, modified ligands, cytokines, and/or immunomodulatory agents can be administered according to the methods of provided herein both to treat an acute episode of disease or to maintain the subject's condition in a non-inflammatory state.

I. Pharmaceutically Acceptable Carriers

The substances provided herein, can be used therapeutically in combination with a pharmaceutically acceptable carrier. By “pharmaceutically acceptable” is meant a material that can be administered to a subject, along with the substance, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

Suitable carriers and their formulations are described in Remington: The Science and Practice of Pharmacy ((19th ed.) ed. A. R. Gennaro, Mack Publishing Company, Easton, Pa. 1995). Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the NF-κB decoy polynucleotide, which matrices are in the form of shaped articles, e.g., films, liposomes or microparticles. It will be apparent to those persons skilled in the art that certain carriers can be more preferable depending upon, for instance, the route of administration and concentration of substance being administered.

Disclosed herein are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a polynucleotide is disclosed and discussed and a number of modifications that can be made are discussed, each and every combination and permutation of the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

It is to be understood that the disclosed methods and compositions are not limited to specific synthetic methods, specified analytical techniques, or to particular reagents unless otherwise specified, and, as such, may vary. It is also to be understood that the terminology used herein is for the purpose of describing particular embodiments only and is not intended to be limiting.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

Mice

C57BL/10 male mice (6-8 weeks old) were used in studies of the acute form of TNBS-colitis and oxazolone-colitis. BALB/c female mice (8-10 weeks old) were used in studies of a chronic form of TNBS-colitis. All mice were obtained from Jackson Laboratories and were maintained in the National Institute of Allergy and Infectious Diseases animal holding facilities. Animal use adhered to National Institutes of Health Laboratory Animal Care Guidelines.

Induction of Colitis

Mice were lightly anesthetized with isoflurane and then administered a haptenating agent (either TNBS or oxazolone dissolved in ethanol) per rectum via a 3.5 F catheter equipped with a 1-ml syringe; the catheter was advanced into the rectum until the tip was 4 cm proximal to the anal verge at which time the haptenating agent was administered in a total volume of 150 μl. To ensure distribution of the haptenating agent within the entire colon and cecum, mice were held in a vertical position for 30 seconds after the intra-rectal injection. Control mice were administered an ethanol solution without haptenating agent using the same technique. 3.75 mg TNBS (Sigma, St. Louis, Mo.) in 50% ethanol was administered for studies of prevention of acute TNBS-colitis, 3 mg TNBS in 45% ethanol for studies of treatment of established acute TNBS-induced colitis and 1.5-2.5 mg TNBS (in increasing doses) in 45% ethanol was administered each week for studies of treatment of chronic TNBS-induced colitis. 6 mg oxazolone (Sigma) in 47.5% ethanol was administered for studies of prevention of oxazolone-colitis.

Preparation of HVJ-E Envelope Vector and its Loading with Polynucleotide

HVJ-E envelope vector was prepared as previously described (39). In brief, suspended Sendai virus (25,600 hemagglutinating units, AnGes MG) was inactivated by β-propiolactone followed by ultraviolet irradiation and purified by column chromatography. The HVJ envelope thus obtained was mixed with 37.5 μl of protamine sulfate (1 mg/ml) and then incubated for 10 min on ice. Insertion of polynucleotide into the vector was accomplished using a packaging technique that allowed direct insertion of polynucleotide into the viral envelope. This involved mixing DNA (1 mg/100 μl) and 13.8 μl of 3% Triton-X with the HVJ envelope and then incubating the resultant mixture for 15 min on ice. Finally, the HVJ envelope-polynucleotide was centrifuged at 15,000 g for 15 min and resuspended in 500 μl of PBS containing 72 μg of protamine sulfate.

Decoy Polynucleotides

75 μg of NF-κB decoy or scrambled double-stranded DNA polynucleotide were administered during each in vivo treatment. The following sequences were used:

| | |
|---|---|
| NF-kB decoy polynucleotide | |
| 5′-CCTTGAAGGGATTTCCCTCC-3′ | (SEQ ID NO:1) |
| and | |
| 3′-GGAACTTCCCTAAAGGGAGG-5′; | (SEQ ID NO:2) |
| scrambled decoy polynucleotide: | |
| 5′-CATGTCGTCACTGCGCTCAT-3′ | (SEQ ID NO:3) |
| and | |
| 3′-GTACAGCAGTGACGCGAGTA-5′. | (SEQ ID NO:4) |

ELISA

Cytokine protein concentrations in culture supernatants were measured by ELISA kits according to the manufacturer's instructions. Isolated colonic lamina propria mononuclear cells were stimulated for 48 h. To determine IFN-γ, IL-4, and IL-13 protein concentrations cells were stimulated with plate-bound anti-CD3-antibody and soluble anti-CD28-antibody (BD Pharmingen, San Diego, Calif.) For measurement of IL-12p70, TNF-α, and TGF-β1 colonic LPMC were stimulated 48 h with Staphylococcus aureus Cowan I (EMD Biosciences, La Jolla, Calif.) and IFN-γ (R&D Systems, Minneapolis, Mn). IL-23 was determined after 48 h stimulation of colonic LPMC with Peptidoglycan (Sigma). ELISA kits for IFN-γ, IL-4, IL-12p70, TNF-α were purchased from BD Pharmingen, for TGF-β1 from Biosource (Rockville, Md.), and IL-23 from eBiosciences (San Diego, Calif.).

Flow Cytometry

Colonic LPMC were stained with Annexin V, Propidium iodide, anti-CD4-antibody (BD Pharmingen). Nonspecific binding of antibodies was blocked by preincubation with Fc-block (BD Pharmingen). Cells were acquired using a Becton Dickinson FACScan and analyzed utilizing FlowJo software.

Collagen Assay

Colons of TNBS-treated mice were harvested on day 49 and homogenized in 0.5M acetic acid containing 1 mg pepsin (at a concentration of 10 mg tissue/10 ml of acetic acid solution), The resulting mixture was then incubated for 24 h at 4° C. with stirring. Colon collagen content was determined by assaying total soluble collagen using the Sircol Collagen Assay kit (Biocolor, Ireland, UK) (65). Acid soluble Type I collagen supplied with the kit was used to generate a standard curve.

Assay of Activated NF-κB Components

Nuclear extracts from colonic LPMC were obtained using the Transfactor Extract Kit (Active Motif, Carlsbad, Calif.). The extracts were then tested for DNA binding activity using the NF-κB TransFactor Kit (BD Clontech, Palo Alto, Calif.) according to the manufacturer's instructions. In brief, nuclear extract (15 to 30 μg) was applied to each well coated with NF-κB consensus polynucleotides and then wells were incubated with specific antibodies for each of the NF-κB subunits followed by horseradish-peroxidase-labeled secondary antibodies (39). After color development with TMB substrate was stopped by adding H₂SO₄, absorbance was measured at 450 nm wavelength.

Histological Examination

Colons were fixed in 10% buffered formalin and embedded in paraffin. Paraffin-embedded colon sections were cut and then stained with H&E or by the Masson's trichrome method. For calculation of inflammation indices or for assessment of fibrosis in treated and control group of mice, the sections were read blindly and evaluated according to a formerly published scoring system (13).

NF-κB Decoy Polynucleotides Block DNA-Binding Activity of NF-κB Family Members

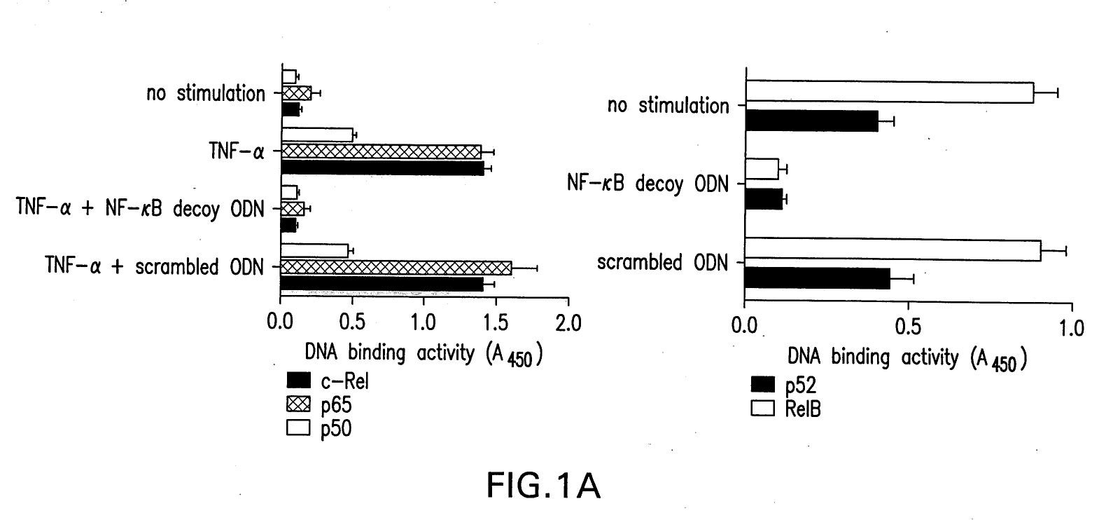

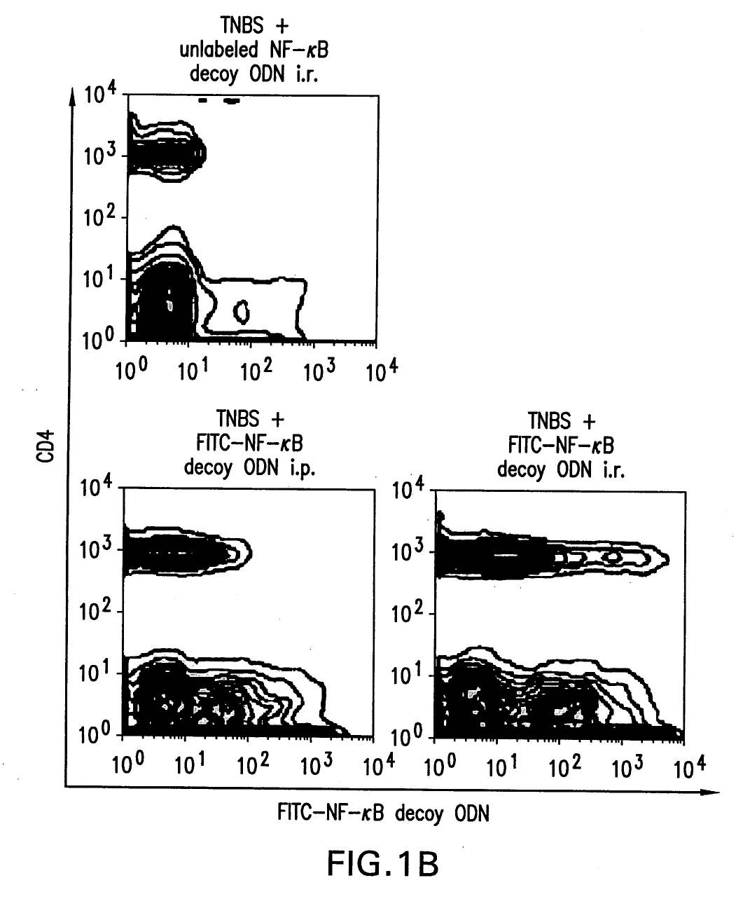

In order to determine the inhibitory effect of NF-κB decoy polynucleotides on each of the family members of NF-κB, the binding activity of all major subunits of NF-κB to a plate-bound consensus binding sequence were measured in the presence and absence of decoy polynucleotides using the TransFactor Assay described above. Nuclear extracts derived from HeLa cells subjected to TNF-α stimulation were the source of the activated NF-κB family members p65, c-Rel, and p50, whereas unstimulated Raji cells (cells in which NF-κB family members are constitutively activated) were the source of Rel B and p52. The inhibitory effect of NF-κB decoy polynucleotide was compared to that of a scrambled polynucleotide, a 22 bp double-stranded DNA sequence not containing any known binding sites for a transcription factor. As shown in FIG. 1A, the ability of all measured NF-κB subunits to bind to the plate-bound consensus sequence was decreased to baseline levels by NF-κB decoy polynucleotide, whereas scrambled polynucleotide showed no inhibitory effects. These findings indicated that NF-κB decoy polynucleotide is a potent inhibitor of all subunits of the NF-κB transcription factor family that might have similar inhibitory effects in vivo.

NF-κB Decoy Polynucleotide Encapsulated in the HVJ-E Viral Envelope is Effectively Transfected into Both CD4+ T Cells and Non-T Cells Present in the Lamina Propria

In further studies mapping the basic characteristics of NF-κB decoy polynucleotide activity, the types of cells undergoing in vivo transfection with decoy polynucleotide following both intra-rectal and intra-peritoneal administration of decoy polynucleotide was determined. To this end, FITC-conjugated NF-κB decoy polynucleotide encapsulated in HVJ-E was administered after TNBS-colitis induction in C57BL/10 mice via an intra-rectal or intra-peritoneal route. The decoy polynucleotide was administered once by the intra-rectal route at four hours after TNBS induction, whereas it was administered three times by the intra-peritoneal route at fours hours, 24 hours and 48 hours after TNBS induction. Then, on day 5 after induction, colonic lamina propria mononuclear cells were isolated and subjected to flow cytometry after staining with PE-labeled anti-CD4-antibody. As shown in FIG. 1B, cells from mice administered unlabeled NF-κB decoy polynucleotide exhibited background FITC fluorescence (except for minimal autofluorescence most probably arising from epithelial cells). In contrast, cells from mice administered labeled decoy polynucleotide exhibited very considerable positive fluorescence in both CD4+ T cells as well as in non-CD4+ cells (a cell population containing APCs, epithelial cells and possibly CD8+ T cells). FITC-positive cells were seen in cells obtained from both mice administered decoy polynucleotide by both the intra-rectal and intra-peritoneal routes, but was somewhat higher in the cells from mice given intra-rectal administration. These studies establish that NF-κB decoy polynucleotide encapsulated in HVJ-E transfect both CD4+ T cells and non-CD4+ cells following both intra-rectal and intra-peritoneal administration.

Administration of NF-κB Decoy Polynucleotide Encapsulated in HVJ-E Prevents Nascent TNBS-Colitis and Reverses Established TNBS-Colitis

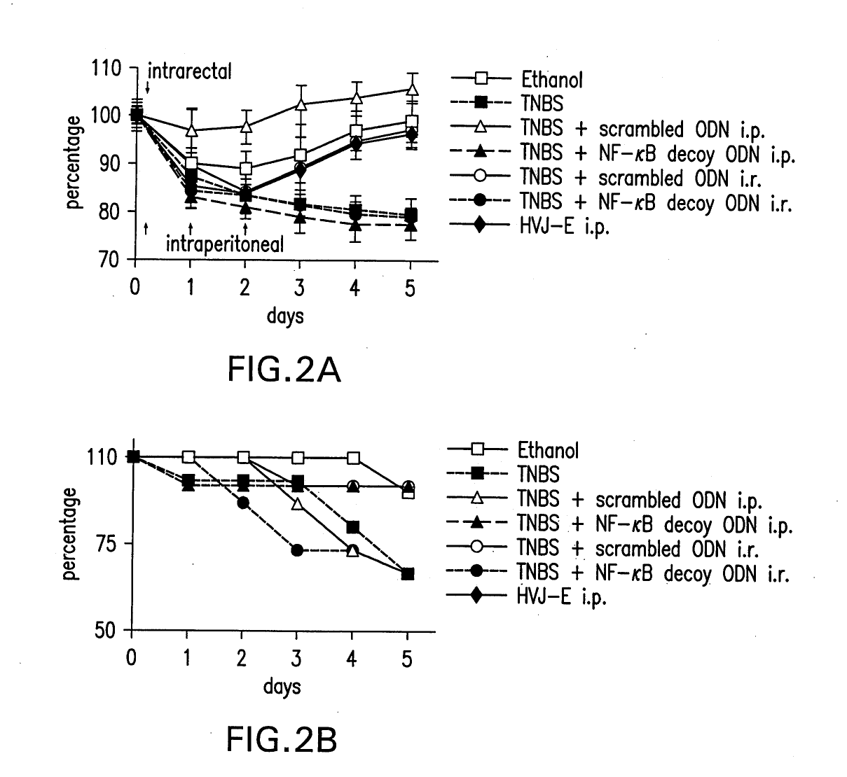

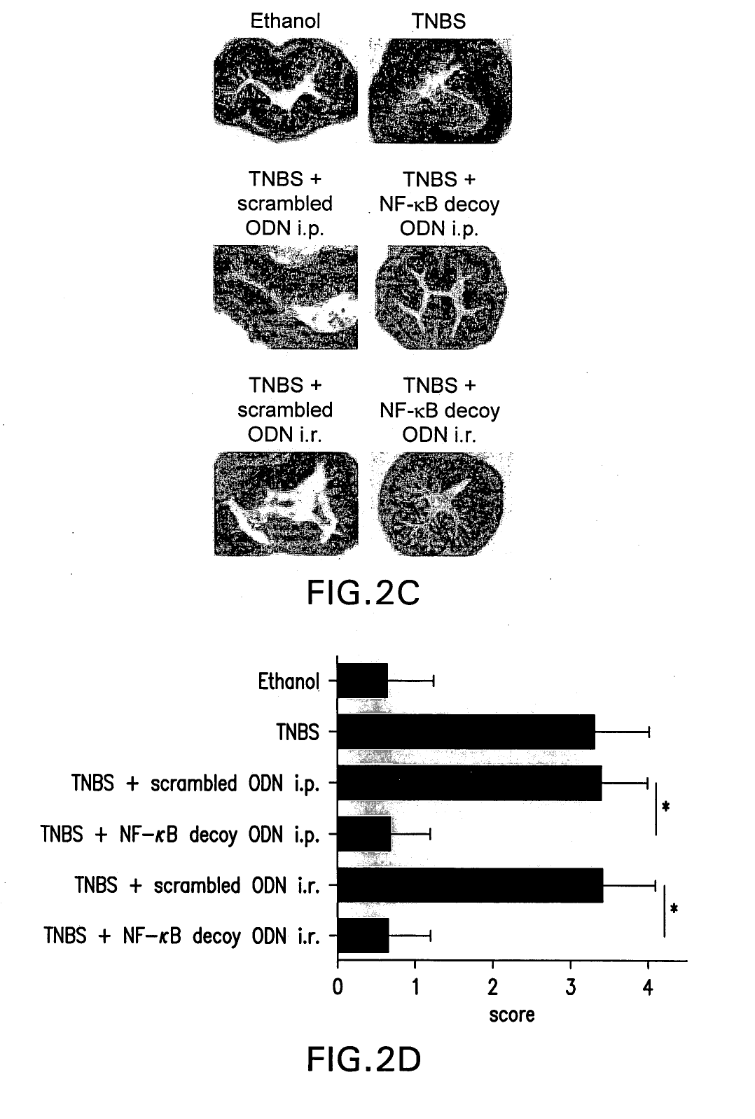

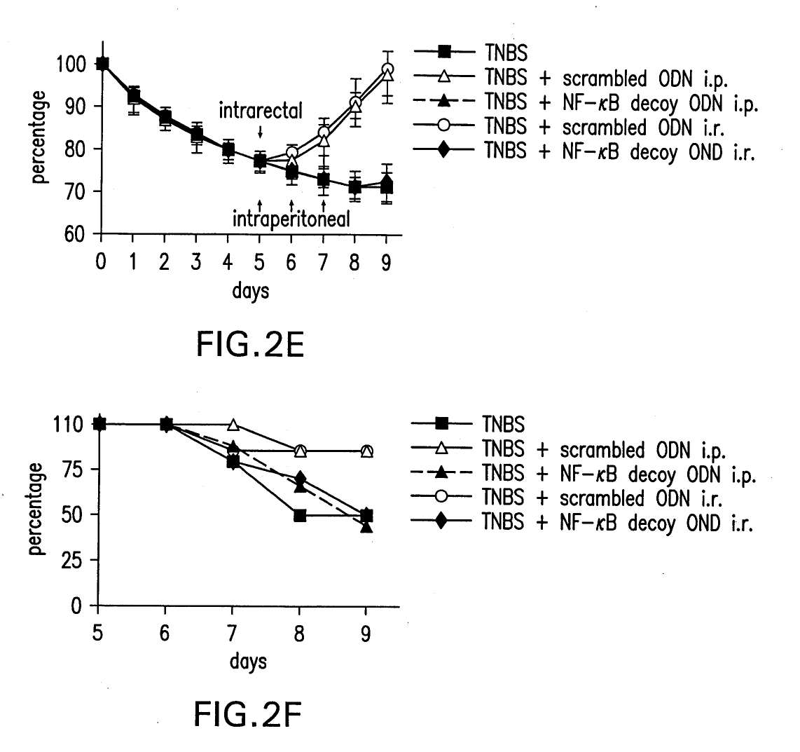

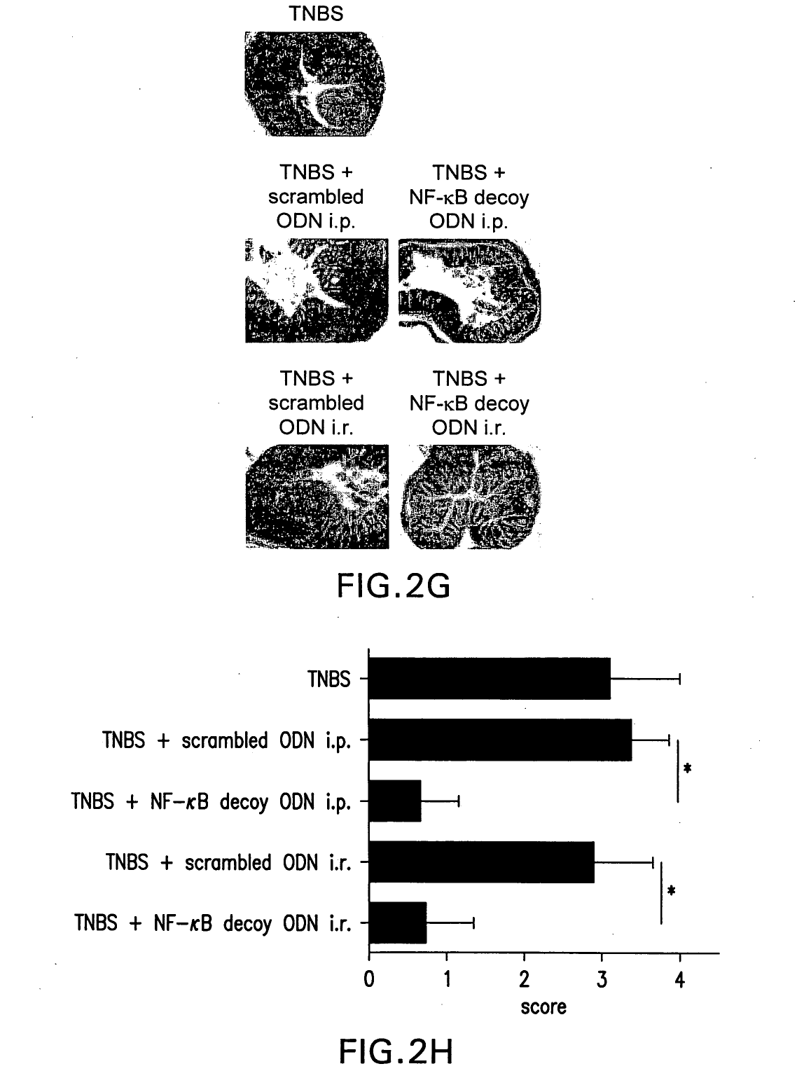

TNBS-colitis induced in SJL/J or C57BL/10 mice is a rapidly evolving transmural colitis that, like Crohn's disease, is a Th1-mediated inflammation dependent on the production of IL-12 (and presumably IL-23). To determine if NF-κB decoy polynucleotide could prevent the development of this colitis in C57BL/10 mice colitis was induced in these mice by intra-rectal instillation of TNBS in ethanol as described above and then, 4 hours later, instilled 75 μg NF-κB decoy polynucleotide or scrambled polynucleotide (encapsulated in HVJ-E). Alternatively these polynucleotide (again, 75 μg packaged in HVJ-E) were administered by intra-peritoneal injection at 4 hours after TNBS administration and again on day 1 and day 2 after TNBS administration. The mice were then monitored by weight loss (or gain), mortality, colon histology and cytokine secretion by cells extracted from tissues and stimulated in vitro. As shown in the weight curves depicted in FIG. 2A and the mortality graph depicted in FIG. 2B, whereas mice administered TNBS/ethanol alone and treated with scrambled polynucleotide exhibited progressive weight loss and high mortality, those treated with NF-κB decoy polynucleotide (either by intra-rectal instillation or intra-peritoneal injections) had a course similar to that observed in control mice treated with ethanol alone. Similarly, the macroscopic appearance of the colons and, as shown in FIGS. 2C and 2D, histological examination of the colons of the NF-κB decoy polynucleotide-treated mice showed no evidence of inflammation whereas the colons of the scrambled polynucleotide-treated mice showed severe inflammation.

A more stringent test of the efficacy of NF-κB decoy polynucleotide as a treatment of TNBS-colitis is whether or not it can reverse already established colitis. To explore this question, TNBS-colitis was again induced in C57BL/10 mice, which were then monitored for weight loss. Ultimately, only those mice that had lost 20% of body weight by the fifth day after colitis induction were selected for treatment with NF-κB decoy polynucleotide (or scrambled polynucleotide). Then, on day 5 after colitis induction, one dose of NF-κB decoy or scrambled polynucleotide (75 μg) was administered by the intra-rectal route or 3 doses of these polynucleotides were administered by the intra-peritoneal route on consecutive days (in both cases encapsulated in HVJ-E), as in the prevention study described above. Care was taken to ensure that the various experimental groups to be compared had lost equivalent amounts of weight. As shown in FIGS. 2E and 2F, untreated mice and mice treated with scrambled polynucleotide continued to lose weight and exhibited a mortality rate at 9 days as high as 50%. In contrast, mice that were treated with NF-κB decoy polynucleotide (by either the intra-rectal or intra-peritoneal route) exhibited a reversal in weight loss and a mortality rate of only 15% at day 9. Furthermore, as shown in FIGS. 2G and 2H, these weight loss and mortality data correlated with histological evaluation of colonic sections. In a similar experiment mice were treated on day 4 and sacrificed on day 7 after TNBS administration with comparable positive treatment effects by NF-κB decoy polynucleotide.

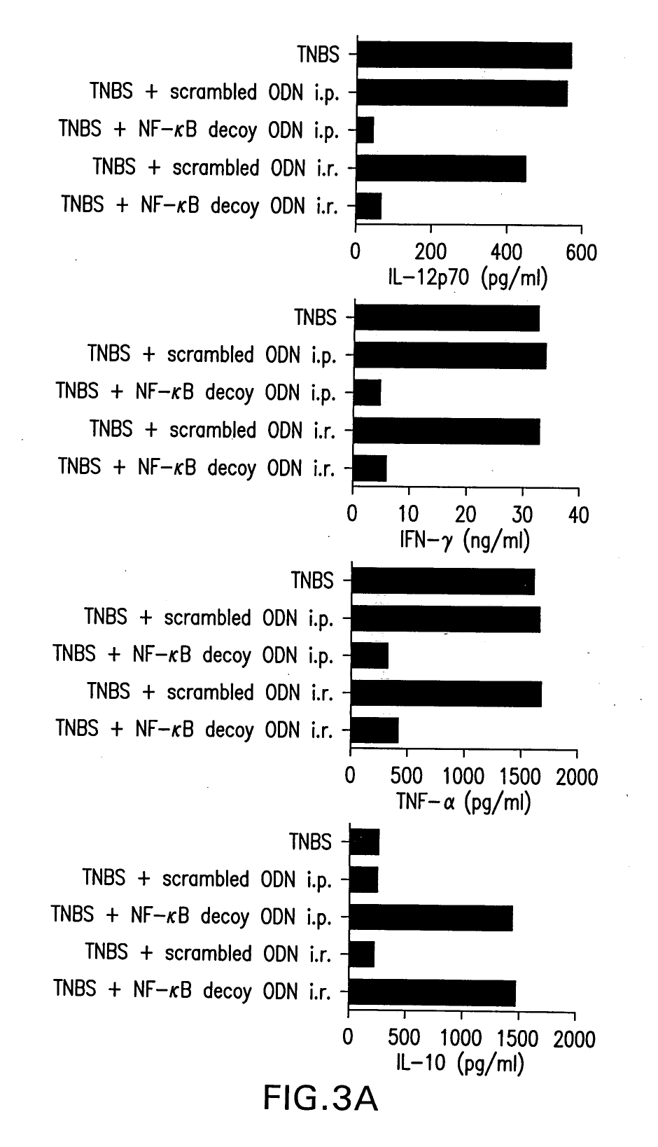

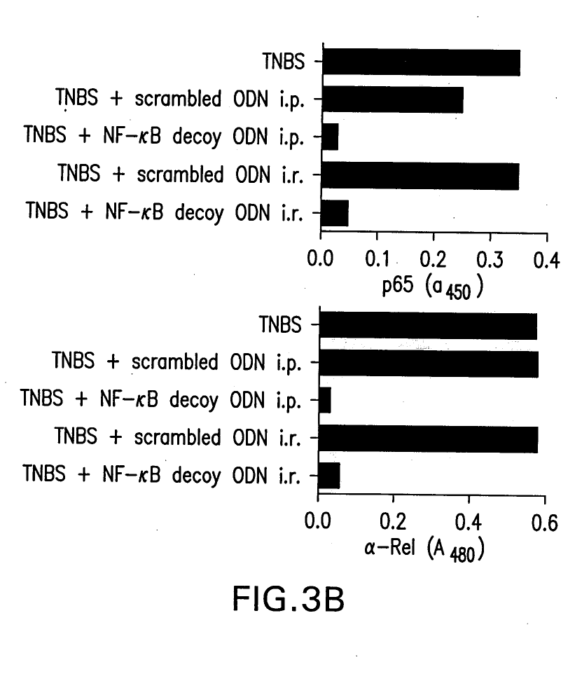

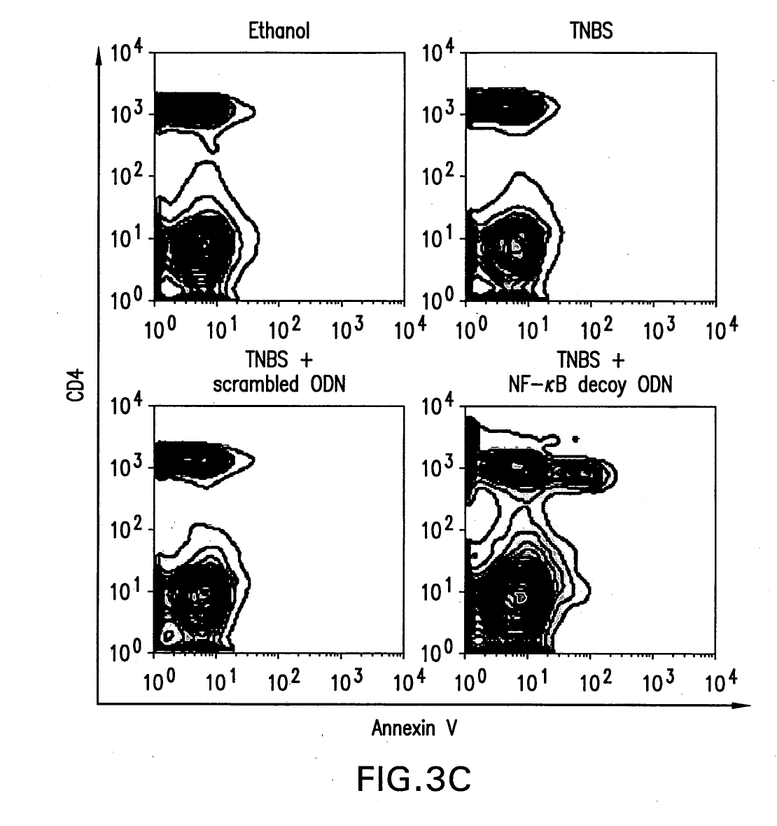

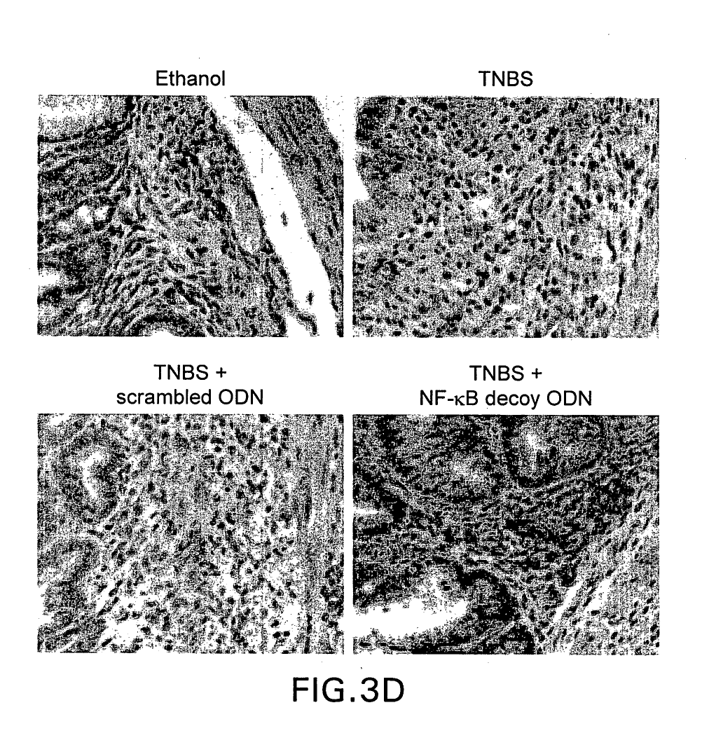

Finally, culture and stimulation of isolated colonic lamina propria mononuclear cells from NF-κB decoy polynucleotide-treated mice, obtained either from the mice administered NF-κB decoy polynucleotide at the time of TNBS-colitis induction to prevent disease or 5 days after induction to treat established disease led to secretion of baseline levels of Th1 cytokines. In contrast, culture and stimulation of cells from untreated or scrambled polynucleotide-treated mice led to secretion of high levels of these cytokines. Thus, as shown in FIG. 3A that depicts the cytokine responses of cells isolated from mice treated with NF-κB decoy polynucleotide with established TNBS-colitis, cells extracted from untreated mice with TNBS-colitis or scrambled polynucleotide-treated with TNBS-colitis, produced high levels of IL-12p70, TNF-α and IFN-γ when appropriately stimulated in vitro whereas cells extracted from the lamina propria of NF-κB decoy polynucleotide-treated mice exhibited levels of cytokine secretion similar to that observed in control ethanol-treated mice. Entirely similar cytokine responses were obtained with cells isolated from mice treated with NF-κB decoy polynucleotide at the time of TNBS-colitis induction (data not shown). Taken together, these data indicate that NF-κB decoy polynucleotide administered intra-rectally or intra-peritoneally is highly effective both in the prevention of nascent TNBS-colitis and in the treatment of established TNBS-colitis. The results of two subsequent studies correlated and expanded on these results. First, studies were conducted to determine if the administration of NF-κB decoy polynucleotide (encapsulated in HVJ-E) results in a persistent inhibition of NF-κB components (p65 and c-Rel) in the context of an existent inflammatory state. Accordingly, nuclear extracts of mononuclear cells isolated from the lamina propria of mice were obtained four days after intra-rectal administration of NF-κB decoy polynucleotide or scrambled polynucleotide treatment (9 days after induction of TNBS-colitis) and then estimated the DNA-binding activity of p65 and c-Rel in the extracts by the extent of binding of these components to plate-bound consensus NF-κB sequences in the TransFactor Assay. As shown in FIG. 3B, extracts of cells from NF-κB decoy polynucleotide-treated mice exhibited greatly decreased p65 and c-Rel binding to the consensus sequence whereas the extracts of scrambled polynucleotide-treated mice exhibited high levels of binding. These data indicate that suppression of binding of NF-κB components occurs during inflammation and persists even after inflammation has subsided.

Second, it was investigated whether or not NF-κB decoy polynucleotide treatment is associated with apoptosis of effector cells in TNBS-colitis as previously shown in treatment of TNBS-induced colitis with anti-IL-12-antibody. To this end, on day 5 after induction of TNBS-colitis (or, on day 5 after administration of ethanol alone) mice were intra-rectally treated NF-κB decoy polynucleotide or scrambled polynucleotide; then, 24 hours later, colonic lamina propria mononuclear cells were isolated and stained with Annexin V for detection of apoptotic cells by flow cytometric analysis. As shown in FIG. 3C, whereas in the untreated or scrambled polynucleotide-treated TNBS-colitis mouse groups, or the ethanol-treated mouse group less than 4% of the CD4+ cells were Annexin V-positive, in the intra-rectal NF-κB decoy polynucleotide-treated TNBS-colitis mouse group 23% of CD4+ cells were Annexin-V-positive. These data show clearly that NF-κB decoy polynucleotide administration causes apoptosis of CD4+ cells in the inflamed gut, suggesting that this form of therapy might have durable effects.

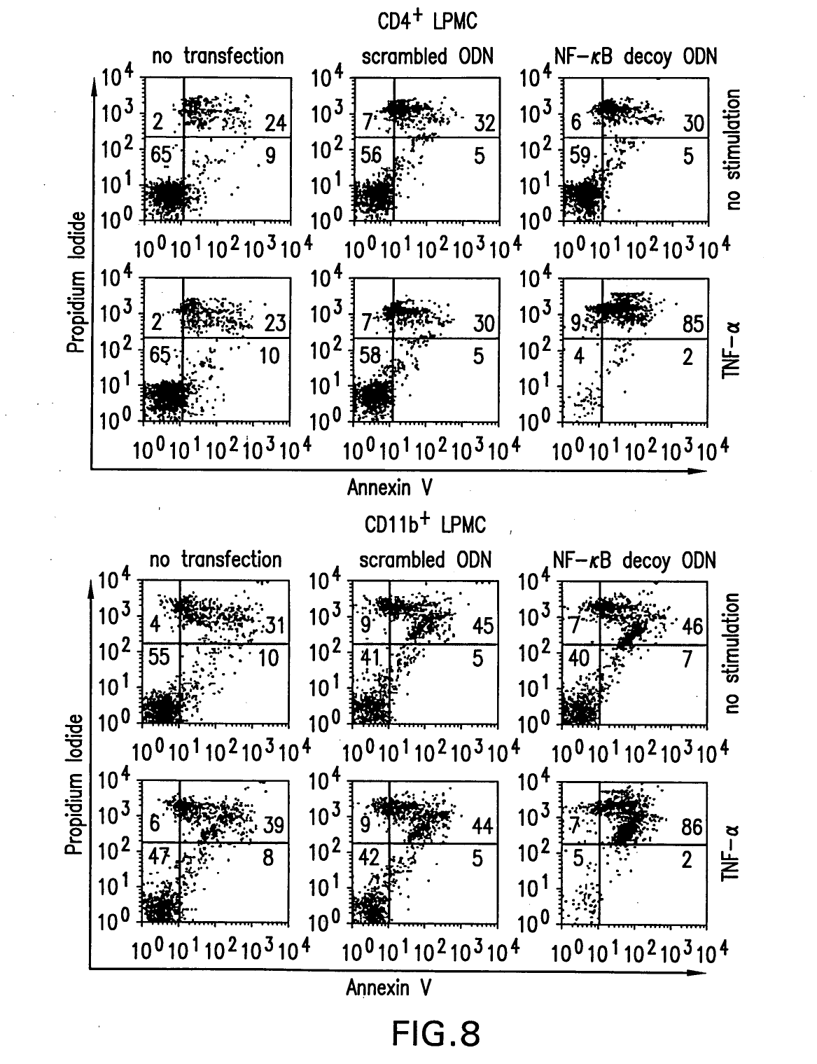

In further studies to identify the cells subject to apoptosis following treatment with NF-κB decoy ODN, we isolated colonic LPMC and separated the latter into CD4⁺ and CD11b⁺ subsets using antibody-coated magnetic beads. After separation, we subjected a portion of the two cell populations to transfection with NF-κB decoy ODN (or scrambled ODN) and then cultured both transfected and untransfected cells with TNF-α for 24 h. Finally, we determined the extent of apoptosis occurring in each cell population by flow cytometric analysis of Annexin V and propidium iodide staining. As shown in FIG. 8, after in vitro culture and transfection of CD4⁺ and CD11b⁺ cells we found that both CD4⁺ and CD11b⁺ cells exhibited greatly enhanced apoptosis after transfection with NF-κB decoy ODN when cultured with TNF-α. The relatively high background apoptosis observed in control cultures is due to the fact, that even after cell purification, the cell populations still contain substantial numbers of colonic epithelial cells and/or granulocytes that undergo cell death during the culture period. These data suggest that NF-κB decoy ODN treatment induces apoptosis in both T cell and APC populations and thus undermines the inflammation-causing immune response at two levels.

Administration of NF-κB Decoy Polynucleotide Packaged in HVJ-E Prevents the Development of Colonic Fibrosis in Chronic TNBS-Colitis.

In the studies described so far the effects of NF-κB decoy polynucleotide were studied in an acute model of TNBS-colitis. Recently, a more chronic form of this type of experimental colitis has been reported in which the colitis is induced in BALB/c mice by weekly intra-rectal administration of increasing doses of TNBS (see Methods) (27). This form of TNBS-colitis in BALB/c mice differs from the more acute form in SJL/J or C57BL/10 mice by the fact that it has both a Th1 and Th2 component. In addition, it leads to the development of fibrosis after the 6th week of TNBS treatment. This model therefore allowed for the determination of the effects of NF-κB decoy polynucleotide on an established colitis (in this case with a somewhat different pathophysiology) and, at the same time to determine the effect of such treatment on the development of cytokine-mediated fibrosis.

In these studies, TNBS was administered by the intra-rectal route each week for 8 weeks to mice. On day 35 after initiation of TNBS administration, mice were assembled into weight-matched sub-groups for various types of treatment. One group of mice were treated with intra-rectal NF-κB decoy polynucleotide packaged in HVJ-E on days 37 and 44 and a second group with intra-peritoneal NF-κB decoy polynucleotide daily on days 37 to 39 and days 44 to 46. A similar regimen was followed for mice treated with scrambled polynucleotide. It should be noted that the TNBS-colitis in these mice did not cause death after week 3, suggesting that at this point the remaining mice had achieved a steady state of inflammation compatible with continued survival.

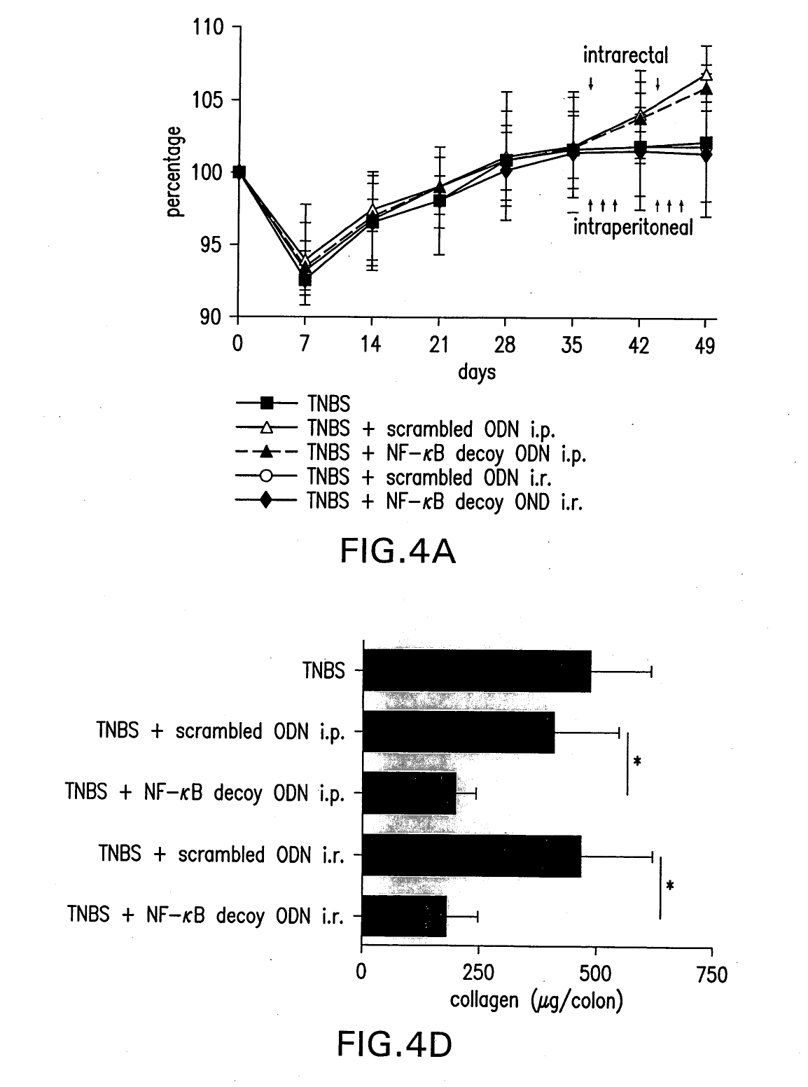

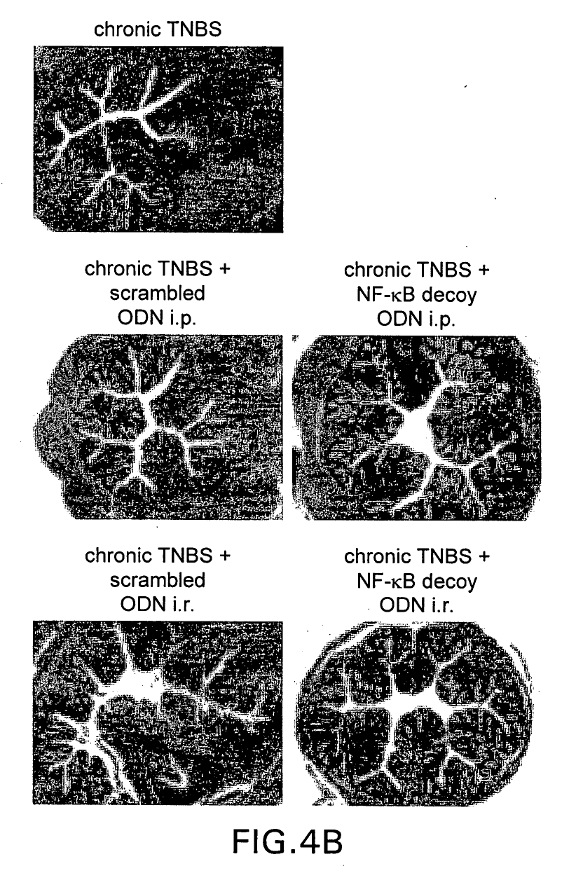

As shown in FIG. 4A, BALB/c mice administered TNBS as described above lost weight during the first 7 days following the initial dose of intra-rectal TNBS but thereafter gained weight and reached their starting weight by day 28 despite re-administration of TNBS. In the following weeks untreated mice and scrambled polynucleotide-treated mice with chronic TNBS-colitis did not gain additional weight whereas NF-κB decoy polynucleotide-treated mice gained additional weight after their first treatment on day 37. As shown in FIG. 4B, these weight changes correlated with the histological evaluation of colonic tissues of the various mouse groups. Untreated and scrambled polynucleotide-treated mice with chronic TNBS-colitis exhibited inflammation of the colonic lamina propria as well as marked thickening of the colon wall whereas NF-κB decoy polynucleotide-treated mice showed comparatively little inflammation of the colonic lamina propria associated with reduced thickness of the colon wall. In addition, as shown in FIG. 4C, while colon tissues stained with the Masson's trichrome technique revealed increased amounts of collagen in the subepithelial and in deeper layers of the colonic lamina propria of untreated mice or scrambled polynucleotide-treated mice, no increase in collagen deposition was observed in NF-κB decoy polynucleotide-treated mice. This reduction in collagen deposition was corroborated by the Sircol collagen assay: the amount of collagen after NF-κB decoy polynucleotide treatment was significantly reduced to almost basal levels (FIG. 4D).

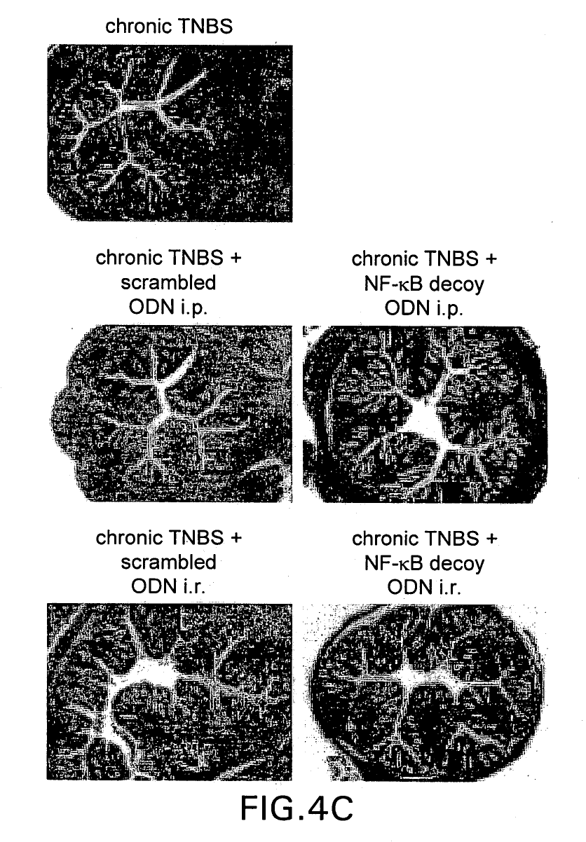

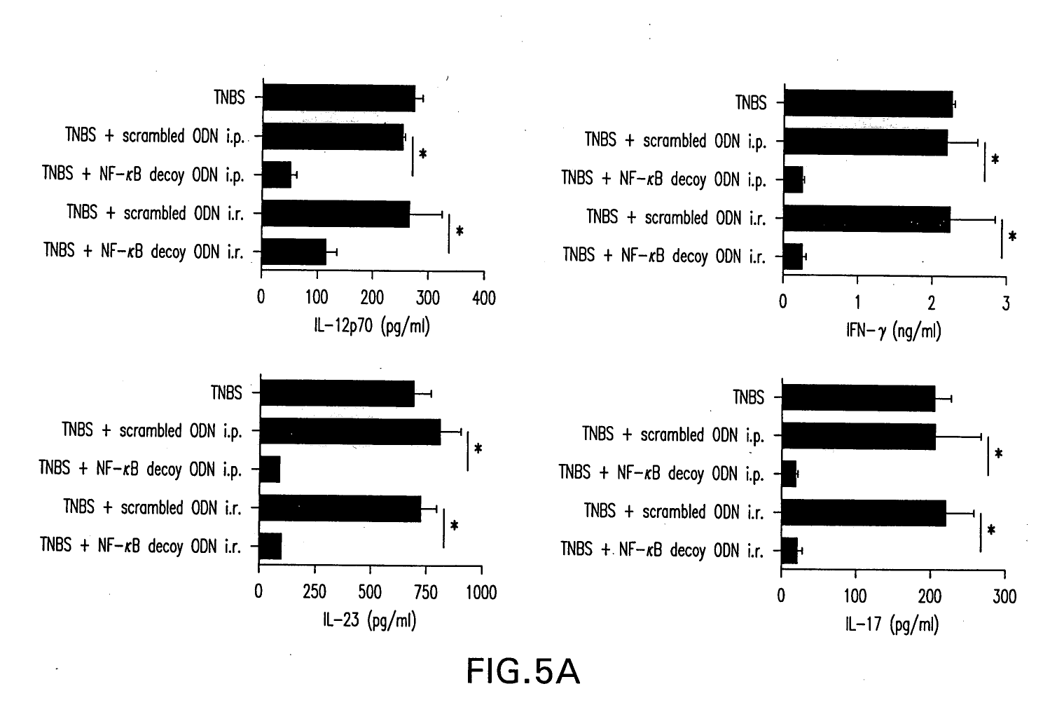

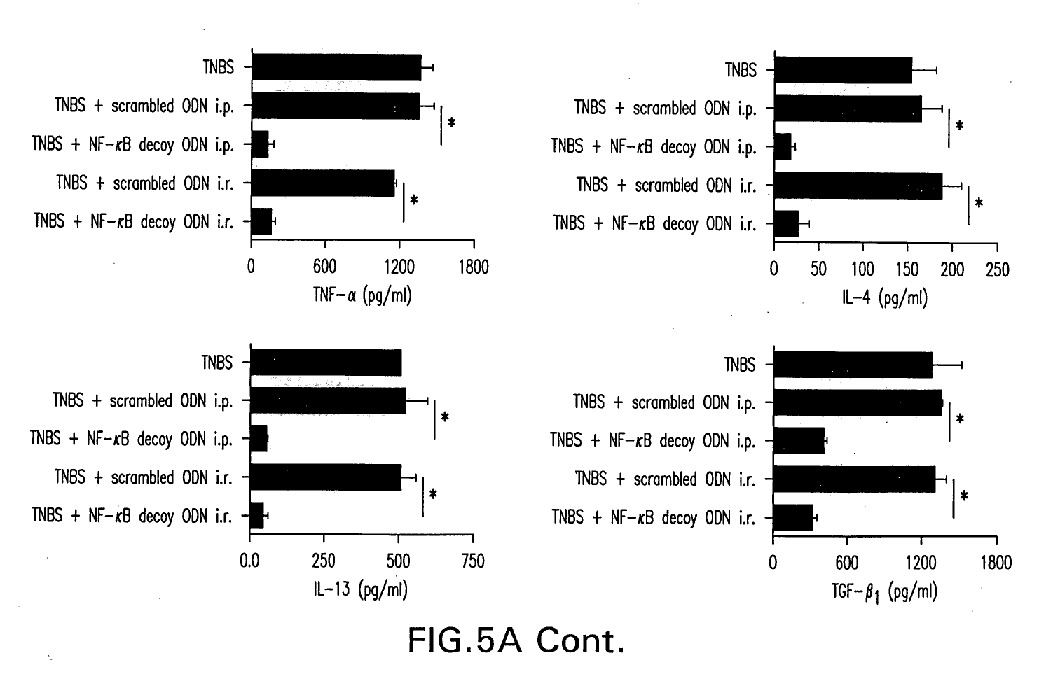

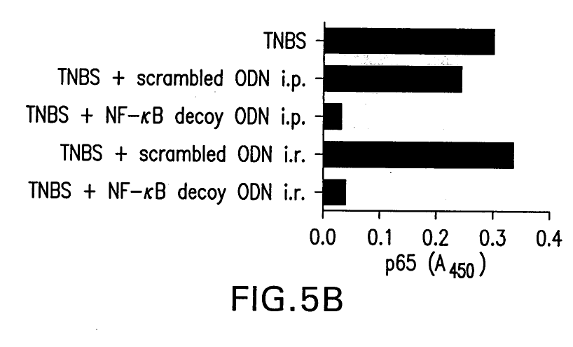

In additional studies of separate groups of mice, the effect of NF-κB polynucleotide treatment of BALB/c mice with chronic TNBS colitis on in vitro cytokine production by lamina propria cells isolated on day 49 was evaluated. As shown in FIG. 5A, cells from untreated and scrambled polynucleotide-treated mice with chronic TNBS-colitis produced elevated levels of Th1 cytokines, such as IL-12p70, IFN-γ, as well as TNF-α, IL-23, and IL-17. In addition, these cells produced increased amounts of Th2 cytokines, including modestly elevated amounts of IL-4 and markedly elevated amounts of IL-13, as well as markedly increased amounts of TGF-β1. In contrast, cells from mice treated with NFκB decoy polynucleotide produced only the basal amount of these cytokines produced by control mice.

Finally, the DNA-binding activity of the NF-κB subunit p65 in nuclear extracts of day 49 lamina propria mononuclear cells from mice treated with scrambled polynucleotide or intra-rectal NF-κB decoy polynucleotide was evaluated. As shown in FIG. 5B, extracts of cells from NF-κB decoy polynucleotide-treated mice once again exhibited low levels of binding to plate-bound NF-κB consensus sequences in the TransFactor assay.

Taken together, these results show that NF-κB decoy polynucleotide can block the development of colitis as well as the development of fibrosis in a chronic model of TNBS-colitis. Given the fact that the development of fibrosis in this model is probable secondary to IL-13 secretion and induction of TGF-β1, this study shows that such treatment also blocks aspects of this inflammation mediated by Th2 cytokines.

Administration of NF-κB Decoy Polynucleotide Packaged in HVJ-E Prevents from Oxazolone-Colitis.

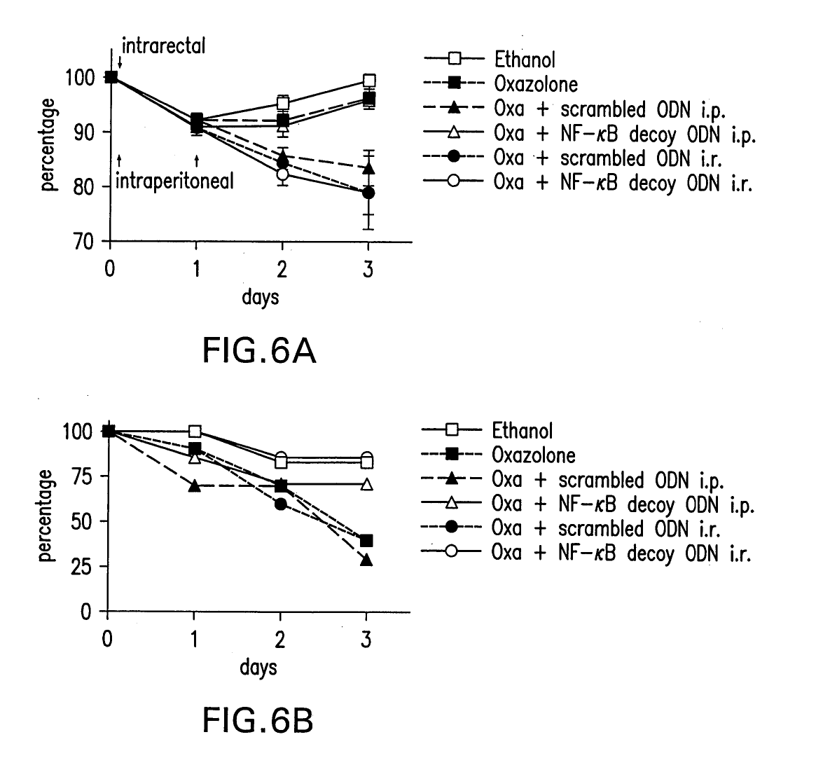

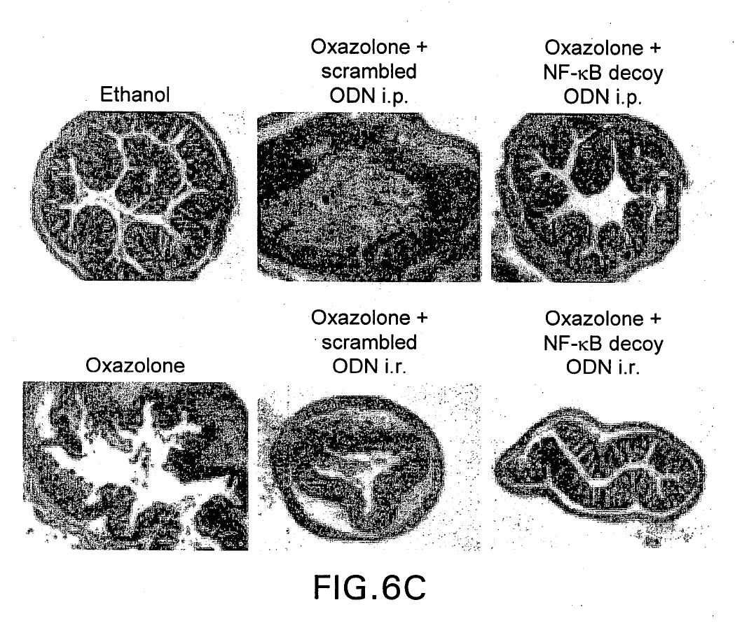

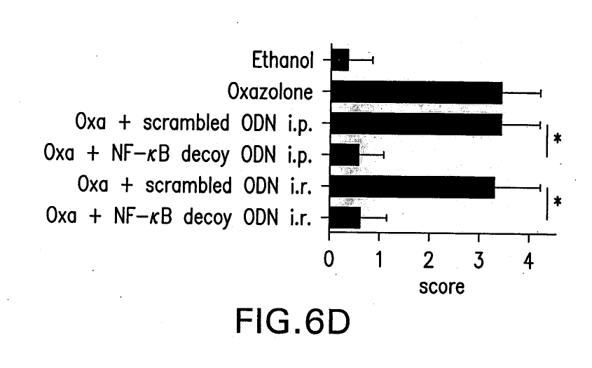

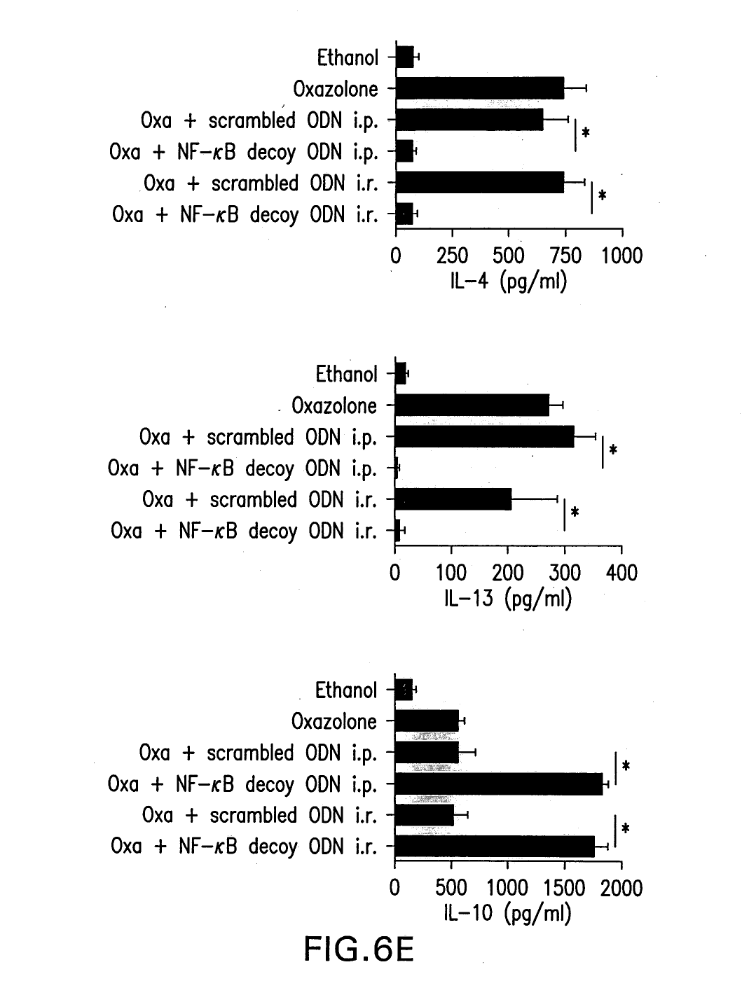

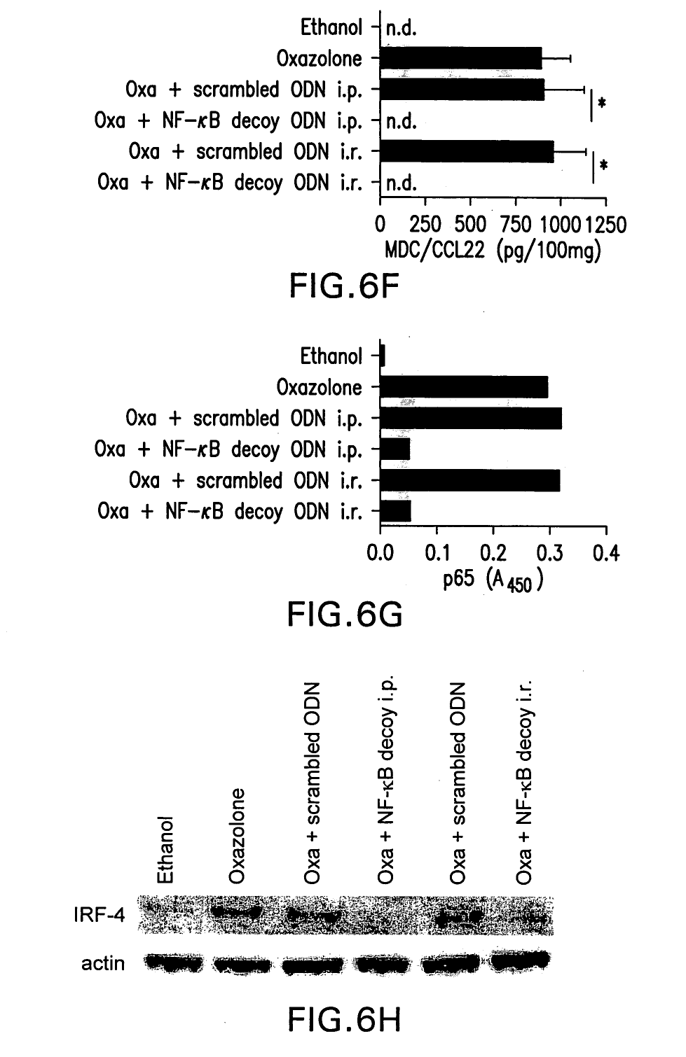

In a further series of studies, the capacity of NF-κB decoy polynucleotide to prevent IBD in a Th2 cytokine-mediated model of hapten-induced colitis that resembles ulcerative colitis, namely oxazolone-colitis was evaluated. Accordingly, oxazolone-colitis was induced in C57BL/10 mice by intra-rectal administration of oxazolone in ethanol, and then the course of colitis was determined in untreated mice or mice treated on the day of oxazolone administration, with either intra-rectal or intra-peritoneal NF-κB decoy polynucleotide or scrambled polynucleotide packaged in HVJ-E. As shown in FIGS. 6A and 6B, untreated mice and mice treated with scrambled polynucleotide exhibited severe weight loss and a 50% mortality rate by day three after induction of oxazolone-colitis, whereas mice treated with NF-κB decoy polynucleotide by either route exhibited a weight equivalent to mice administered ethanol alone and a greatly reduced mortality. Moreover, as shown in FIG. 6C, these clinical findings correlated with histological examination of colons from mice in the various groups: untreated and scrambled polynucleotide-treated mice exhibited high levels of inflammation associated with extensive epithelial cell ulceration whereas NF-κB decoy polynucleotide-treated mice exhibited virtually no inflammation. Finally, as shown in FIG. 6D, colonic lamina propria cells extracted on day 3 after induction of colitis from untreated and scrambled polynucleotide-treated mice produced high levels of both IL-4 and IL-13 upon stimulation. Corresponding cells from NF-κB decoy polynucleotide produced basal amounts of these cytokines. In the same fashion, secretion of a Th2-associated chemokine, MDC/CCL22, was increased in ex vivo cultures of colons from untreated and scrambled polynucleotide-treated mice, but was undetectable in cultures of colons from NF-κB decoy polynucleotide-treated mice.

Once again, as shown in FIG. 6E, nuclear extracts of colonic lamina propria mononuclear cells from untreated or scrambled polynucleotide-treated mice exhibited high levels of binding of p65 to plate-bound NF-κB consensus sequences whereas extracts of cells from NF-κB decoy polynucleotide exhibited low levels of binding. Thus, NF-κB decoy polynucleotide packaged in HVJ-E provides effective means to prevent NF-κB signaling in the context of a Th2 inflammation. These studies therefore show that NF-κB decoy polynucleotide has profound effects on a Th2 model of colonic inflammation as well as on Th1 models.

Intra-Rectal Administration of NF-κB Decoy Polynucleotide Suppresses Local (Mucosal) NF-κB Activity but not NF-κB Activity in a Distant Organ.

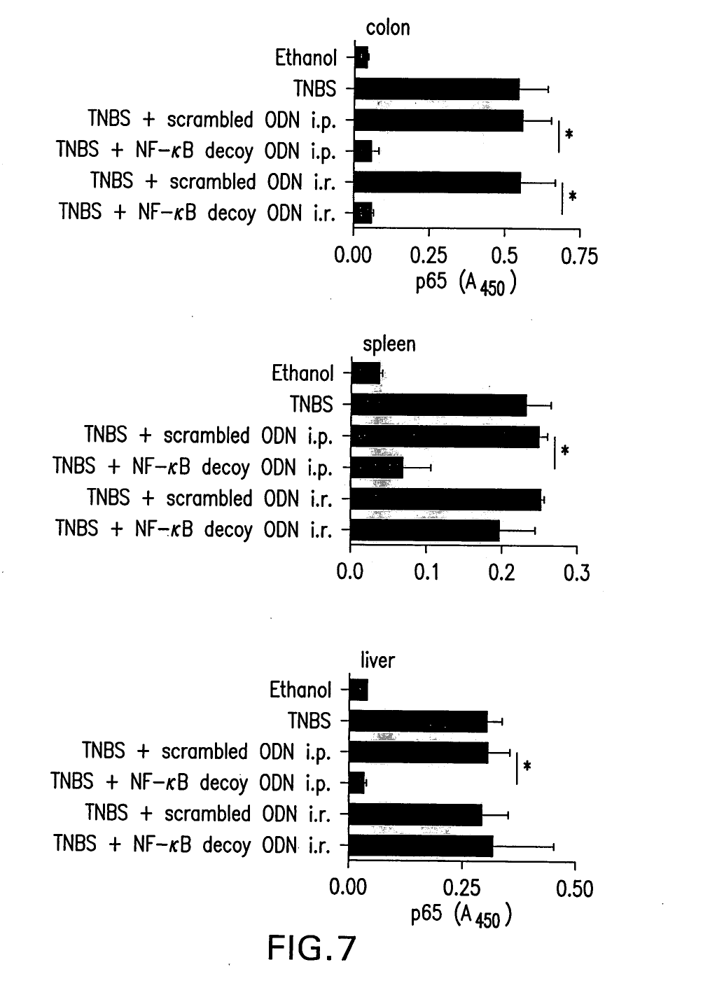

In a final series of studies the effects of intra-rectal and intra-peritoneal NF-κB decoy polynucleotide administration on NF-κB activation outside of the colon was determined. Accordingly, mice administered intra-rectal TNBS to induced TNBS-colitis were treated with NF-κB decoy polynucleotide by intra-rectal (4 h) or intra-peritoneal (4 h, day 1, day 2) routes. Then, on day 5 after TNBS induction, nuclear extracts from mononuclear cells isolated from the colonic lamina propria, spleen and liver were obtained. The extracts were then subjected to a TransFactor assay to determine p65 DNA binding activity. As shown in FIG. 7, whereas intra-peritoneal administration of NF-κB decoy polynucleotide led to decreased p65 activity in cells from all three organs, intra-rectal administration led to greatly decreased activity in colonic lamina propria cells, but no decreased activity in spleen or hepatic cells. These studies thus show that local administration of NF-κB decoy polynucleotide has little effect on extra-intestinal mononuclear cells.

Throughout this application, various publications are referenced. The disclosures of these publications in their entireties are hereby incorporated by reference into this application in order to more fully describe the state of the art to which this invention pertains.

### REFERENCES

1. Podolsky, D. K. 2002. Inflammatory bowel disease. N Engl J Med 347:417-429.
2. Bouma, G., and Strober, W. 2003. The immunological and genetic basis of inflammatory bowel disease. Nat Rev Immunol 3:521-533.
3. Sartor, R. B. 2004. Therapeutic manipulation of the enteric microflora in inflammatory bowel diseases: antibiotics, probiotics, and prebiotics. Gastroenterology 126:1620-1633.
4. Maeda, S., Hsu, L. C., Liu, H., Bankston, L. A., Iimura, M., Kagnoff, M. F., Eckmann, L., and Karin, M. 2005. Nod2 mutation in Crohn's disease potentiates NF-kappaB activity and IL-1beta processing. Science 307:734-738.
5. Strober, W., Fuss, I. J., and Blumberg, R. S. 2002. The immunology of mucosal models of inflammation. Annu Rev Immunol 20:495-549.
6. Uhlig, H. H., and Powrie, F. 2003. Dendritic cells and the intestinal bacterial flora: a role for localized mucosal immune responses. J Clin Invest 112:648-651.
7. Monteleone, G., Biancone, L., Marasco, R., Morrone, G., Marasco, O., Luzza, F., and Pallone, F. 1997. Interleukin 12 is expressed and actively released by Crohn's disease intestinal lamina propria mononuclear cells. Gastroenterology 112:1169-1178.
8. Mannon, P. J., Fuss, I. J., Mayer, L., Elson, C. O., Sandborn, W. J., Present, D., Dolin, B., Goodman, N., Groden, C., Hornung, R. L., et al. 2004. Anti-interleukin-12 antibody for active Crohn's disease. N Engl J Med 351:2069-2079.
9. Fuss, I. J., Heller, F., Boirivant, M., Leon, F., Yoshida, M., Fichtner-Feigl, S., Yang, Z., Exley, M., Kitani, A., Blumberg, R. S., et al. 2004. Nonclassical CD1d-restricted NK T cells that produce IL-13 characterize an atypical Th2 response in ulcerative colitis. J Clin Invest 113:1490-1497.
10. Schmidt, C., Giese, T., Ludwig, B., Mueller-Molaian, I., Marth, T., Zeuzem, S., Meuer, S. C., and Stallmach, A. 2005. Expression of interleukin-12-related cytokine transcripts in inflammatory bowel disease: elevated interleukin-23p19 and interleukin-27p28 in Crohn's disease but not in ulcerative colitis. Inflamm Bowel Dis 11:16-23.
11. Neurath, M. F., Fuss, I., Kelsall, B. L., Stuber, E., and Strober, W. 1995. Antibodies to interleukin 12 abrogate established experimental colitis in mice. J Exp Med 182:1281-1290.
12. Kitani, A., Fuss, I. J., Nakamura, K., Schwartz, O. M., Usui, T., and Strober, W. 2000. Treatment of experimental (Trinitrobenzene sulfonic acid) colitis by intranasal administration of transforming growth factor (TGF)-beta1 plasmid: TGF-beta1-mediated suppression of T helper cell type 1 response occurs by interleukin (IL)-10 induction and IL-12 receptor beta2 chain downregulation. J Exp Med 192:41-52.
13. Fuss, I. J., Marth, T., Neurath, M. F., Pearlstein, G. R., Jain, A., and Strober, W. 1999. Anti-interleukin 12 treatment regulates apoptosis of Th1 T cells in experimental colitis in mice. Gastroenterology 117:1078-1088.
14. Boirivant, M., Fuss, I. J., Chu, A., and Strober, W. 1998. Oxazolone colitis: A murine model of T helper cell type 2 colitis treatable with antibodies to interleukin 4. J Exp Med 188:1929-1939.
15. Heller, F., Fuss, I. J., Nieuwenhuis, E. E., Blumberg, R. S., and Strober, W. 2002. Oxazolone colitis, a Th2 colitis model resembling ulcerative colitis, is mediated by IL-13-producing NK-T cells. Immunity 17:629-638.
16. Neurath, M. F., Pettersson, S., Meyer zum Buschenfelde, K. H., and Strober, W. 1996. Local administration of antisense phosphorothioate oligonucleotides to the p65 subunit of NF-kappa B abrogates established experimental colitis in mice. Nat Med 2:998-1004.
17. Neurath, M. F., Fuss, I., Schurmann, G., Pettersson, S., Arnold, K., Muller-Lobeck, H., Strober, W., Herfarth, C., and Buschenfelde, K. H. 1998. Cytokine gene transcription by NF-kappa B family members in patients with inflammatory bowel disease. Ann N Y Acad Sci 859:149-159.
18. Schreiber, S., Nikolaus, S., and Hampe, J. 1998. Activation of nuclear factor kappa B inflammatory bowel disease. Gut 42:477-484.
19. Bantel, H., Berg, C., Vieth, M., Stolte, M., Kruis, W., and Schulze-Osthoff, K. 2000. Mesalazine inhibits activation of transcription factor NF-kappaB in inflamed mucosa of patients with ulcerative colitis. Am J Gastroenterol 95:3452-3457.
20. Plevy, S. E., Gemberling, J. H., Hsu, S., Domer, A. J., and Smale, S. T. 1997. Multiple control elements mediate activation of the murine and human interleukin 12 p40 promoters: evidence of functional synergy between C/EBP and Rel proteins. Mol Cell Biol 17:4572-4588.
21. Becker, C., Wirtz, S., Blessing, M., Pirhonen, J., Strand, D., Bechthold, O., Frick, J., Galle, P. R., Autenrieth, I., and Neurath, M. F. 2003. Constitutive p40 promoter activation and IL-23 production in the terminal ileum mediated by dendritic cells. J Clin Invest 112:693-706.
22. Hilliard, B. A., Mason, N., Xu, L., Sun, J., Lamhamedi-Cherradi, S. E., Liou, H. C., Hunter, C., and Chen, Y. H. 2002. Critical roles of c-Rel in autoimmune inflammation and helper T cell differentiation. J Clin Invest 110:843-850.
23. Zhang, Z., Andoh, A., Yasui, H., Inatomi, O., Hata, K., Tsujikawa, T., Kitoh, K., Takayanagi, A., Shimizu, N., and Fujiyama, Y. 2005. Interleukin-1beta and tumor necrosis factor-alpha upregulate interleukin-23 subunit p19 gene expression in human colonic subepithelial myofibroblasts. Int J Mol Med 15:79-83.
24. Das, J., Chen, C. H., Yang, L., Cohn, L., Ray, P., and Ray, A. 2001. A critical role for NF-kappa B in GATA3 expression and TH2 differentiation in allergic airway inflammation. Nat Immunol 2:45-50.
25. Schnare, M., Barton, G. M., Holt, A. C., Takeda, K., Akira, S., and Medzhitov, R. 2001. Toll-like receptors control activation of adaptive immune responses. Nat Immunol 2:947-950.
26. Pahl, A., Zhang, M., Kuss, H., Szelenyi, I., and Brune, K. 2002. Regulation of IL-13 synthesis in human lymphocytes: implications for asthma therapy. Br J Pharmacol 135:1915-1926.
27. Lawrance, I. C., Wu, F., Leite, A. Z., Willis, J., West, G. A., Fiocchi, C., and Chakravarti, S. 2003. A murine model of chronic inflammation-induced intestinal fibrosis down-regulated by antisense NF-kappa B. Gastroenterology 125:1750-1761.
28. Li, Q., and Verma, I. M. 2002. NF-kappaB regulation in the immune system. Nat Rev Immunol 2:725-734.
29. Kotani, H., Nakajima, T., Lai, S., Morishita, R., and Kaneda, Y. 2004. The HVJ-envelope as an innovative vector system for cardiovascular disease. Curr Gene Ther 4:183-194.
30. Morishita, R., Sugimoto, T., Aoki, M., Kida, I., Tomita, N., Moriguchi, A., Maeda, K., Sawa, Y., Kaneda, Y., Higaki, J., et al. 1997. In vivo transfection of cis element “decoy” against nuclear factor-kappaB binding site prevents myocardial infarction. Nat Med 3:894-899.
31. Tomita, N., Ogihara, T., and Morishita, R. 2003. Transcription factors as molecular targets: molecular mechanisms of decoy ODN and their design. Curr Drug Targets 4:603-608.
32. Gambari, R. 2004. New trends in the development of transcription factor decoy (TFD) pharmacotherapy. Curr Drug Targets 5:419-430.
33. Kaneda, Y., Nakajima, T., Nishikawa, T., Yamamoto, S., Ikegami, H., Suzuki, N., Nakamura, H., Morishita, R., and Kotani, H. 2002. Hemagglutinating virus of Japan (HVJ) envelope vector as a versatile gene delivery system. Mol Ther 6:219-226.
34. Shimamura, M., Sato, N., Oshima, K., Aoki, M., Kurinami, H., Waguri, S., Uchiyama, Y., Ogihara, T., Kaneda, Y., and Morishita, R. 2004. Novel therapeutic strategy to treat brain ischemia: overexpression of hepatocyte growth factor gene reduced ischemic injury without cerebral edema in rat model. Circulation 109:424-431.
35. Saeki, Y., Matsumoto, N., Nakano, Y., Mori, M., Awai, K., and Kaneda, Y. 1997. Development and characterization of cationic liposomes conjugated with HVJ (Sendai virus): reciprocal effect of cationic lipid for in vitro and in vivo gene transfer. Hum Gene Ther 8:2133-2141.
36. Stehlik, C., de Martin, R., Binder, B. R., and Lipp, J. 1998. Cytokine induced expression of porcine inhibitor of apoptosis protein (iap) family member is regulated by NF-kappa B. Biochem Biophys Res Commun 243:827-832.
37. Tang, G., Minemoto, Y., Dibling, B., Purcell, N. H., Li, Z., Karin, M., and Lin, A. 2001. Inhibition of JNK activation through NF-kappaB target genes. Nature 414:313-317.
38. Papa, S., Zazzeroni, F., Pham, C. G., Bubici, C., and Franzoso, G. 2004. Linking JNK signaling to NF-kappaB: a key to survival. J Cell Sci 117:5197-5208.
39. Watanabe, T., Kitani, A., Murray, P. J., and Strober, W. 2004. NOD2 is a negative regulator of Toll-like receptor 2-mediated T helper type 1 responses. Nat Immunol 5:800-808.
40. Lawrence, T., Gilroy, D. W., Colville-Nash, P. R., and Willoughby, D. A. 2001. Possible new role for NF-kappaB in the resolution of inflammation. Nat Med 7:1291-1297.
41. Tomita, T., Takeuchi, E., Tomita, N., Morishita, R., Kaneko, M., Yamamoto, K., Nakase, T., Seki, H., Kato, K., Kaneda, Y., et al. 1999. Suppressed severity of collagen-induced arthritis by in vivo transfection of nuclear factor kappaB decoy oligodeoxynucleotides as a gene therapy. Arthritis Rheum 42:2532-2542.
42. Suzuki, J., Morishita, R., Amano, J., Kaneda, Y., and Isobe, M. 2000. Decoy against nuclear factor-kappa B attenuates myocardial cell infiltration and arterial neointimal formation in murine cardiac allografts. Gene Ther 7:1847-1852.
43. Ogushi, I., limuro, Y., Seki, E., Son, G., Hirano, T., Hada, T., Tsutsui, H., Nakanishi, K., Morishita, R., Kaneda, Y., et al. 2003. Nuclear factor kappa B decoy oligodeoxynucleotides prevent endotoxin-induced fatal liver failure in a murine model. Hepatology 38:335-344.
44. Kim, K. W., Cho, M. L., Park, M. K., Yoon, C. H., Park, S. H., Lee, S. H., and Kim, H. Y. 2005. Increased interleukin-17 production via a phosphoinositide 3-kinase/Akt and nuclear factor kappaB-dependent pathway in patients with rheumatoid arthritis. Arthritis Res Ther 7:R139-148.
45. Tominaga, N., Ohkusu-Tsukada, K., Udono, H., Abe, R., Matsuyama, T., and Yui, K. 2003. Development of Th1 and not Th2 immune responses in mice lacking IFN-regulatory factor-4. Int Immunol 15: 1-10.
46. Hu, C. M., Jang, S. Y., Fanzo, J. C., and Pernis, A. B. 2002. Modulation of T cell cytokine production by interferon regulatory factor-4. J Biol Chem 277:49238-49246.
47. Kim, S. W., Choi, S. C., Choi, E. Y., Kim, K. S., Oh, J. M., Lee, H. J., Oh, H. M., Kim, S., Oh, B. S., Kimm, K. C., et al. 2004. Catalposide, a compound isolated from catalpa ovata, attenuates induction of intestinal epithelial proinflammatory gene expression and reduces the severity of trinitrobenzene sulfonic Acid-induced colitis in mice. Inflamm Bowel Dis 10:564-572.
48. Matsumoto, S., Nagaoka, M., Hara, T., Kimura-Takagi, I., Mistuyama, K., and Ueyama, S. 2004. Fucoidan derived from Cladosiphon okamuranus Tokida ameliorates murine chronic colitis through the down-regulation of interleukin-6 production on colonic epithelial cells. Clin Exp Immunol 136:432-439.
49. Sugimoto, K., Hanai, H., Tozawa, K., Aoshi, T., Uchijima, M., Nagata, T., and Koide, Y. 2002. Curcumin prevents and ameliorates trinitrobenzene sulfonic acid-induced colitis in mice. Gastroenterology 123:1912-1922.
50. Hollenbach, E., Neumann, M., Vieth, M., Roessner, A., Malfertheiner, P., and Naumann, M. 2004. Inhibition of p38 MAP kinase- and RICK/NF-kappaB-signaling suppresses inflammatory bowel disease. Faseb J 18:1550-1552.
51. Segain, J. P., Raingeard de la Bletiere, D., Sauzeau, V., Bourreille, A., Hilaret, G., Cario-Toumaniantz, C., Pacaud, P., Galmiche, J. P., and Loirand, G. 2003. Rho kinase blockade prevents inflammation via nuclear factor kappa B inhibition: evidence in Crohn's disease and experimental colitis. Gastroenterology 124:1180-1187.
52. Takagi, T., Naito, Y., Tomatsuri, N., Handa, O., Ichikawa, H., Yoshida, N., and Yoshikawa, T. 2002. Pioglitazone, a PPAR-gamma ligand, provides protection from dextran sulfate sodium-induced colitis in mice in association with inhibition of the NF-kappaB-cytokine cascade. Redox Rep 7:283-289.
53. Herfarth, H., Brand, K., Rath, H. C., Rogler, G., Scholmerich, J., and Falk, W. 2000. Nuclear factor-kappa B activity and intestinal inflammation in dextran sulphate sodium (DSS)-induced colitis in mice is suppressed by gliotoxin. Clin Exp Immunol 120:59-65.
54. Zhou, Y., Su, Y., Li, B., Liu, F., Ryder, J. W., Wu, X., Gonzalez-DeWhitt, P. A., Gelfanova, V., Hale, J. E., May, P. C., et al. 2003. Nonsteroidal anti-inflammatory drugs can lower amyloidogenic Abeta42 by inhibiting Rho. Science 302:1215-1217.
55. Hirooka, Y., Shimokawa, H., and Takeshita, A. 2004. Rho-kinase, a potential therapeutic target for the treatment of hypertension. Drug News Perspect 17:523-527.
56. Hashimoto, K., Peebles, R. S., Jr., Sheller, J. R., Jarzecka, K., Furlong, J., Mitchell, D. B., Hartert, T. V., and Graham, B. S. 2002. Suppression of airway hyperresponsiveness induced by ovalbumin sensitisation and RSV infection with Y-27632, a Rho kinase inhibitor. Thorax 57:524-527.
57. Russell, F. D., and Molenaar, P. 2004. Investigation of signaling pathways that mediate the inotropic effect of urotensin-II in human heart. Cardiovasc Res 63:673-681.
58. Kweon, Y. O., Paik, Y. H., Schnabl, B., Qian, T., Lemasters, J. J., and Brenner, D. A. 2003. Gliotoxin-mediated apoptosis of activated human hepatic stellate cells. J Hepatol 39:38-46.
59. Waring, P., Sjaarda, A., and Lin, Q. H. 1995. Gliotoxin inactivates alcohol dehydrogenase by either covalent modification or free radical damage mediated by redox cycling. Biochem Pharmacol 49:1195-1201.
60. Vulcano, M., Albanesi, C., Stoppacciaro, A., Bagnati, R., D'Amico, G., Struyf, S., Transidico, P., Bonecchi, R., Del Prete, A., Allavena, P., et al. 2001. Dendritic cells as a major source of macrophage-derived chemokine/CCL22 in vitro and in vivo. Eur J Immunol 31:812-822.
61. Nakayama, T., Hieshima, K., Nagakubo, D., Sato, E., Nakayama, M., Kawa, K., and Yoshie, O. 2004. Selective induction of Th2-attracting chemokines CCL17 and CCL22 in human B cells by latent membrane protein 1 of Epstein-Barr virus. J Virol 78:1665-1674.
62. Berin, M. C., Dwinell, M. B., Eckmann, L., and Kagnoff, M. F. 2001. Production of MDC/CCL22 by human intestinal epithelial cells. Am J Physiol Gastrointest Liver Physiol 280:G1217-1226.
63. Elewaut, D., Shaikh, R. B., Hammond, K. J., De Winter, H., Leishman, A. J., Sidobre, S., Turovskaya, O., Prigozy, T. I., Ma, L., Banks, T. A., et al. 2003. NIK-dependent RelB activation defines a unique signaling pathway for the development of V alpha 14i NKT cells. J Exp Med 197:1623-1633.
64. Lee, C. G., Homer, R. J., Zhu, Z., Lanone, S., Wang, X., Koteliansky, V., Shipley, J. M., Gotwals, P., Noble, P., Chen, Q., et al. 2001. Interleukin-13 induces tissue fibrosis by selectively stimulating and activating transforming growth factor beta(1). J Exp Med 194:809-821.
65. Kitani, A., Fuss, I., Nakamura, K., Kumaki, F., Usui, T., and Strober, W. 2003. Transforming growth factor (TGF)-beta1-producing regulatory T cells induce Smad-mediated interleukin 10 secretion that facilitates coordinated immunoregulatory activity and amelioration of TGF-beta1-mediated fibrosis. J Exp Med 198:1179-1188.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating or preventing inflammatory bowel disease (IBD) in a subject comprising administering to the subject a therapeutically effective amount of a composition comprising an NF-κB decoy nucleic acid.

2. The method of claim 1, wherein the inflammatory bowel disease is ulcerative colitis.

3. The method of claim 1, wherein the inflammatory bowel disease is Crohn's disease.

4. The method of claim 1, wherein one or more NF-κB subunits selected from the group consisting of NF-κB1 (p50, p105), NF-κB2 (p52, p100), RelA (p65), RelB, or c-Rel bind the NF-κB decoy nucleic acid.

5. The method of claim 1, wherein the NF-κB decoy nucleic acid comprises the nucleic acid sequence SEQ ID NO:1.

6. The method of claim 1, wherein the NF-κB decoy nucleic acid is DNA.

7. The method of claim 1, wherein the NF-κB decoy nucleic acid is RNA

8. The method of claim 1, wherein the NF-κB decoy nucleic acid is single stranded.

9. The method of claim 1, wherein the NF-κB decoy nucleic acid is double stranded.

10. The method of claim 1, wherein the NF-κB decoy nucleic acid is linear.

11. The method of claim 1, wherein the NF-κB decoy nucleic acid is circular

12. The method of claim 1, wherein the NF-κB decoy nucleic acid is a double stranded oligodeoxynucleotide.

13. The method of claim 1, wherein the NF-κB decoy nucleic acid comprises the NF-κB-consensus binding sequence.

14. The method of claim 1, wherein the NF-κB decoy nucleic acid comprises the NF-κB-binding domain in the promoters of genes encoding 12-Lipoxygenase, 5-Lipoxygenase, (I) collagen, B7.1 (CD80), Bax, Bcl-2, b-Interferon, CCL28, CCL5, CD154, CD40, CD95 (Fas), Claudin-2, Collagenase 1, COX-2, CXCL 11, Eotaxin, Fas-Ligand, Fibronectin, Fractalkine, G-CSF, GM-CSF, HGF/SF, IAPs, ICAM-1, IFN-g, IL-1 receptor antagonist, IL-11, IL-12 (p40), IL-12 (p35), IL-13, IL-15, IL17, IL23 (p19), IL-1a, IL-1b, IL-2, IL-6, IL-8, iNOS, IP-10, IRF-1, IRF-2, IRF-4, RF-7, MadCAM-1, MCP-1/JE, MIP-1a,b (LAG-1), MIP-3alpha, MIG, Nod2, Phospholipase A2, RANTES, RICK, TNFa, TNF-Receptor (p75/80,CD120B), or VCAM-1.

15. The method of claim 1, wherein the NF-κB decoy nucleic acid is in a vector.

16. The method of claim 1, wherein the NF-κB decoy nucleic acid is packaged in a liposome.

17. The method of claim 1, wherein the NF-κB decoy nucleic acid is packaged in a viral envelope.

18. The method of claim 17, wherein the viral envelope is an HVJ-envelope.

19. The method of claim 1, wherein the NF-κB decoy nucleic acid is packaged in a chimeric liposome comprising viral envelope-derived fusion (fusigenic) proteins.

20. The method of claim 19, wherein the chimeric liposome is a HVJ liposome complex.

21. The method of claim 1, wherein the composition is administered to the subject systemically.

22. The method of claim 1, wherein the composition is administered to the subject intraperitoneally.

23. The method of claim 1, wherein the composition is administered to the subject intrarectally.

24. The method of claim 1, wherein the NF-κB decoy nucleic acid is delivered to the nucleus of epithelial cells, antigen presenting cells, B-cells, T-cells, macrophages, monocytes, eosinophils, fibroblasts, or neutrophils.

25. The method of claim 24, wherein the composition is delivered to the cells by electroporation or sonoporation.

26. The method of claim 1, wherein a Th1 inflammatory response is ameliorated in the subject.

27. The method of claim 1, wherein a Th2 inflammatory response is ameliorated in the subject.


SEQUENCE LISTING (32 sequences)

SEQ ID NO: 1 - DNA, 20 bp, Artificial Sequence
  Description of Artificial Sequence; note = synthetic construct
ccttgaagggatttccctcc

SEQ ID NO: 2 - DNA, 20 bp, Artificial Sequence
  Description of Artificial Sequence; note = synthetic construct
ggaacttccctaaagggagg

SEQ ID NO: 3 - DNA, 20 bp, Artificial Sequence
  Description of Artificial Sequence; note = synthetic construct
catgtcgtcactgcgctcat

SEQ ID NO: 4 - DNA, 20 bp, Artificial Sequence
  Description of Artificial Sequence; note = synthetic construct
gtacagcagtgacgcgagta

SEQ ID NO: 5 - DNA, 9 bp, Artificial Sequence
  Description of Artificial Sequence; note = synthetic construct
gggatttcc

SEQ ID NO: 6 - DNA, 12 bp, Artificial Sequence
  Description of Artificial Sequence; note = synthetic construct
ggggtatttccc

SEQ ID NO: 7 - DNA, 11 bp, Artificial Sequence
  Description of Artificial Sequence; note = synthetic construct
ggggtatttcc

SEQ ID NO: 8 - DNA, 9 bp, Artificial Sequence
  Description of Artificial Sequence; note = synthetic construct
ggggatccc

SEQ ID NO: 9 - DNA, 10 bp, Artificial Sequence
  Description of Artificial Sequence; note = synthetic construct
gggrnwttcc

SEQ ID NO: 10 - DNA, 10 bp, Artificial Sequence
  Description of Artificial Sequence; note = synthetic construct
gggactttcc

SEQ ID NO: 11 - DNA, 10 bp, Artificial Sequence
  Description of Artificial Sequence; note = synthetic construct
ggggattccc

SEQ ID NO: 12 - DNA, 10 bp, Artificial Sequence
  Description of Artificial Sequence; note = synthetic construct
gggaaattcc

SEQ ID NO: 13 - DNA, 10 bp, Artificial Sequence
  Description of Artificial Sequence; note = synthetic construct
ggggatttcc

SEQ ID NO: 14 - DNA, 10 bp, Artificial Sequence
  Description of Artificial Sequence; note = synthetic construct
gggattttcc

SEQ ID NO: 15 - DNA, 10 bp, Artificial Sequence
  Description of Artificial Sequence; note = synthetic construct
ggggatttcc

SEQ ID NO: 16 - DNA, 10 bp, Artificial Sequence
  Description of Artificial Sequence; note = synthetic construct
gggagattcc

SEQ ID NO: 17 - DNA, 10 bp, Artificial Sequence
  Description of Artificial Sequence; note = synthetic construct
gggggcttcc

SEQ ID NO: 18 - DNA, 10 bp, Artificial Sequence
  Description of Artificial Sequence; note = synthetic construct
ggggctttcc

SEQ ID NO: 19 - DNA, 10 bp, Artificial Sequence
  Description of Artificial Sequence; note = synthetic construct
ggggtttccc

SEQ ID NO: 20 - DNA, 10 bp, Artificial Sequence
  Description of Artificial Sequence; note = synthetic construct
gggatttccc

SEQ ID NO: 21 - DNA, 10 bp, Artificial Sequence
  Description of Artificial Sequence; note = synthetic construct
gggaattccc

SEQ ID NO: 22 - DNA, 10 bp, Artificial Sequence
  Description of Artificial Sequence; note = synthetic construct
gggatttcac

SEQ ID NO: 23 - DNA, 10 bp, Artificial Sequence
  Description of Artificial Sequence; note = synthetic construct
gggaactacc

SEQ ID NO: 24 - DNA, 10 bp, Artificial Sequence
  Description of Artificial Sequence; note = synthetic construct
gggaaagtac

SEQ ID NO: 25 - DNA, 10 bp, Artificial Sequence
  Description of Artificial Sequence; note = synthetic construct
gggdnwttcc

SEQ ID NO: 26 - DNA, 16 bp, Artificial Sequence
  Description of Artificial Sequence; note = synthetic construct
ccttgaagggatttcc

SEQ ID NO: 27 - DNA, 16 bp, Artificial Sequence
  Description of Artificial Sequence; note = synthetic construct
gggatttccgtctaga

SEQ ID NO: 28 - DNA, 28 bp, Artificial Sequence
  Description of Artificial Sequence; note = synthetic construct
gggatttccaggatcagaagggatttcc

SEQ ID NO: 29 - DNA, 32 bp, Artificial Sequence
  Description of Artificial Sequence; note = synthetic construct
gggatttccagtactggcagggatttccctcc

SEQ ID NO: 30 - DNA, 32 bp, Artificial Sequence
  Description of Artificial Sequence; note = synthetic construct
agtcgggatttccgtccatgatcgggatttcc

SEQ ID NO: 31 - DNA, 36 bp, Artificial Sequence
  Description of Artificial Sequence; note = synthetic construct
ctgagggatttccgactagtcatgggatttccatac

SEQ ID NO: 32 - DNA, 47 bp, Artificial Sequence
  Description of Artificial Sequence; note = synthetic construct
gggatttccagtacatgcggggatttccgatctgataggggatttcc